(19) United States
(12) Patent Application Publication
(10) Pub. No.: US 2020/0246385 A1
(43) Pub. Date: Aug. 6, 2020

(54) MICRO-ORGANOIDS, AND METHODS OF MAKING AND USING THE SAME

(71) Applicant: Celularity Inc., Warren, NJ (US)

(72) Inventors: Robert J. HARIRI, Bernardsville, NJ (US); Mohit BHATIA, Manalapan, NJ (US); Jia-Lun WANG, Cherry Hill, NJ (US); Wolfgang HOFGARTNER, Florham Park, NJ (US); Qian YE, Livingston, NJ (US)

(73) Assignee: Celularity Inc., Warren, NJ (US)

(21) Appl. No.: 16/854,608

(22) Filed: Apr. 21, 2020

Related U.S. Application Data
(63) Continuation of application No. 15/117,669, filed on Aug. 9, 2016, now abandoned.
(60) Provisional application No. 61/938,536, filed on Feb. 11, 2014.

(51) Int. Cl. A61K 35/17; A61K 35/30; C12N 5/00; A61K 35/36; A61K 35/32; A61K 35/50; A61K 35/44; A61K 35/407; A61K 35/39; A61K 35/28

(52) U.S. Cl. CPC A61K 35/17; A61K 35/30; C12N 5/0062; A61K 35/36; A61K 35/28; A61K 35/50; A61K 35/44; A61K 35/407; A61K 35/39; A61K 35/32

(57) ABSTRACT

Provided herein are micro-organoids, referred to herein as Functional Physiological Units (FPUs), that are capable of replacing or augmenting one or more physiological functions in an individual, which are useful in the treatment of individuals lacking, or suffering a deficit in, said physiological function.

### 1 FIELD

Provided herein are micro-organoids, referred to herein as Functional Physiological Units (FPUs), that are capable of replacing or augmenting one or more physiological functions in an individual, which are useful in the treatment of individuals lacking, or suffering a deficit in, said physiological function.

### 2 BACKGROUND

There exists a great medical need for the replacement of the physiological functionality of diseased, damaged or surgically removed tissues. Provided herein are micro-organoids (Functional Physiological Units), and methods of making and using the same, which fulfill this need.

### 3 SUMMARY

Throughout, Functional Physiological Units are referred to in the plural; however, any characteristics or combinations thereof described herein may, in certain embodiments, be applicable to individual FPUs as well.

Provided herein are micro-organoids, which are, or comprise, a functional physiological unit of one or more organs. In one aspect, provided herein are Functional Physiological Units (FPUs), wherein said FPUs comprise an isolated extracellular matrix (ECM) and at least one type of cell, wherein said FPUs perform at least one function of an organ, or a tissue from an organ, where said FPUs are less than about 1000 microliters in volume, wherein said at least one function of an organ or tissue from an organ is production of a protein, growth factor, cytokine, interleukin, or small molecule characteristic of at least one cell type from said organ or tissue, and wherein said FPUs are in administrable or injectable form. The FPU may perform said at least one function of an organ or a tissue from an organ at any point in its lifespan; that is, once produced, an FPU may perform said one or more function immediately, or upon culturing, or upon differentiation of one of said at least one type of cell (e.g., stem or progenitor cells) at some point during the life of the FPU.

In various embodiments, said FPUs are less than about 100 microliters in volume; less than about 1 microliter in volume; less than about 100 picoliters in volume; or less than about 10 picoliters in volume. In other various embodiments, said FPUs are less than about 10 millimeters along the longest axis; less than about 1 millimeter along the longest axis; or less than about 100 μM along the longest axis. In other various embodiments, said FPUs comprise no more than about 10⁵ cells; no more than about 10⁴ cells; no more than about 10³ cells; or no more than about 10² cells.

In another embodiment, said FPUs comprise at least one channel traversing said FPUs, wherein said channel facilitates diffusion of nutrients and/or oxygen to said cells.

In a specific embodiment of any of the embodiments herein, said FPUs additionally comprise a synthetic matrix. In a more specific embodiment, said synthetic matrix stabilizes the three-dimensional structure of said FPUs. In certain specific embodiments, said synthetic matrix comprises a polymer or a thermoplastic. In certain specific embodiments, said synthetic matrix is a polymer or a thermoplastic. In more specific embodiments, said thermoplastic is polycaprolactone, polylactic acid, polybutylene terephthalate, polyethylene terephthalate, polyethylene, polyester, polyvinyl acetate, or polyvinyl chloride. In certain other specific embodiments, said polymer is polyvinylidine chloride, poly(o-carboxyphenoxy)-p-xylene) (poly(o-CPX)), poly(lactide-anhydride) (PLAA), n-isopropyl acrylamide, acrylamide, pent erythritol diacrylate, polymethyl acrylate, carboxymethylcellulose, or poly(lactic-co-glycolic acid) (PLGA). In certain other specific embodiments, said polymer is polyacrylamide.

In a specific embodiment, said extracellular matrix is placental extracellular matrix, e.g., extracellular matrix is telopeptide placental collagen. In a more specific embodiment, said extracellular matrix is placental extracellular matrix comprising base-treated and/or detergent treated Type I telopeptide placental collagen that has not been chemically modified or contacted with a protease, wherein said ECM comprises less than 5% fibronectin or less than 5% laminin by weight; between 25% and 92% Type I collagen by weight; between 2% and 50% Type III collagen; between 2% and 50% type IV collagen by weight; and/or less than 40% elastin by weight. In a more specific embodiment, said telopeptide placental collagen is base-treated, detergent treated Type I telopeptide placental collagen, wherein said collagen has not been chemically modified or contacted with a protease, and wherein said composition comprises less than 1% fibronectin by weight; less than 1% laminin by weight; between 74% and 92% Type I collagen by weight; between 4% and 6% Type III collagen by weight; between 2% and 15% type IV collagen by weight; and/or less than 12% elastin by wei ght. In certain embodiments, said ECM is crosslinked or stabilized. In certain other embodiments, said ECM is combined with a polymer that stabilizes the three-dimensional structure of said FPU.

In certain embodiments, any of the FPUs described herein have, or substantially have, the shape of a rectangular block, a cube, a sphere, a spheroid, a rod, a cylinder, trapezoid, pyramid, or a torus. In certain other embodiments, any of the FPUs described herein comprises voids, communicating with the surface of said FPUs, large enough to permit entry or exit of cells. In certain other embodiments, any of the FPUs described herein comprises voids, communicating with the surface of said FPUs, wherein said voids are not large enough to permit entry or exit of cells.

In certain specific embodiments, said cells in said FPUs comprise natural killer (NK) cells, e.g., CD56⁺ CD16⁻ placental intermediate natural killer (PiNK) cells. In certain other specific embodiments, said FPUs comprise dendritic cells.

In certain specific embodiments, said FPUs comprise thymocytes. In certain other embodiments, said FPUs comprise any combination of, or all of, thymocytes, lymphoid cells, epithelial reticular cells, and thymic stromal cells.

In certain other specific embodiments, said FPUs comprise thyroid follicular cells. In certain other embodiments, said FPUs comprise cells that express thyroglobulin. In certain other specific embodiments, said FPUs additionally comprise thyroid epithelial cells and parafollicular cells.

In certain specific embodiments, said FPUs comprise stem cells and/or progenitor cells, or are generated in part or whole using stem cells and/or progenitor cells. In specific embodiments, said stem cells or progenitor cells are embryonic stem cells, embryonic germ cells, induced pluripotent stem cells, mesenchymal stem cells, bone marrow-derived mesenchymal stem cells, bone marrow-derived mesenchymal stromal cells, tissue plastic-adherent placental stem cells (PDACs), umbilical cord stem cells, amniotic fluid stem cells, amnion derived adherent cells (AMDACs), osteogenic placental adherent cells (OPACs), adipose stem cells, limbal stem cells, dental pulp stem cells, myoblasts, endothelial progenitor cells, neuronal stem cells, exfoliated teeth derived stem cells, hair follicle stem cells, dermal stem cells, parthenogenically derived stem cells, reprogrammed stem cells, amnion derived adherent cells, or side population stem cells. In certain other specific embodiments, said FPUs comprise hematopoietic stem cells or hematopoietic progenitor cells. In certain other specific embodiments, said FPUs comprise tissue culture plastic-adherent CD34⁻, CD10⁺, CD105⁺, and CD200⁺ placental stem cells. In a more specific embodiment, said placental stem cells are additionally one or more of CD45⁻, CD80⁻, CD86⁻, or CD90⁺. In a more specific embodiment, said placental stem cells are additionally CD45⁻, CD80⁻, CD86⁻, and CD90⁺. In another more specific embodiment, said placental stem cells, when said FPUs are implanted into a recipient, suppress an immune response in said recipient, e.g., locally within said recipient.

In certain other specific embodiments, any of the FPUs described herein comprise differentiated cells. In more specific embodiments, said differentiated cells comprise one or more of:
endothelial cells, epithelial cells, dermal cells, endodermal cells, mesodermal cells, fibroblasts, osteocytes, chondrocytes, natural killer cells, dendritic cells, hepatic cells, pancreatic cells, or stromal cells;
salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells,
somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell,
blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells,
respiratory tract ciliated cells, oviduct ciliated cell, uterine endometrial ciliated cells, rete testis ciliated cells, ductulus efferens ciliated cells, ciliated ependymal cells,
epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells,
surface epithelial cells of stratified squamous epithelium, basal cell of epithelia, urinary epithelium cells,
auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of car, type I taste bud cells,
cholinergic neural cells, adrenergic neural cells, peptidergic neural cells,
inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells,
astrocytes, neurons, oligodendrocytes, spindle neurons,
anterior lens epithelial cells, crystallin-containing lens fiber cells,
hepatocytes, adipocytes, white fat cells, brown fat cells, liver lipocytes,
kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells,
ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells,
red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands,
reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, B cells, natural killer cells,
melanocytes, retinal pigmented epithelial cells,
oogonia/oocytes, spermatids, spermatocytes, spermatogonium cells, spermatozoa, ovarian follicle cells, Sertoli cells, thymus epithelial cell, and/or interstitial kidney cells.

In certain other specific embodiments, said cells are primary culture cells. In another specific embodiment, said cells are cells that have been cultured in vitro. In certain other specific embodiments, said cells have been genetically engineered to produce a protein or polypeptide not naturally produced by the cells, or have been genetically engineered to produce a protein or polypeptide in an amount greater than that naturally produced by the cells. In specific embodiments, said protein or polypeptide is a cytokine or a peptide comprising an active part thereof In more specific embodiments, said cytokine is one or more of adrenomedullin (AM), angiopoietin (Ang), bone morphogenetic protein (BMP), brain-derived neurotrophic factor (BDNF), epidermal growth factor (EGF), erythropoietin (Epo), fibroblast growth factor (FGF), glial cell line-derived neurotrophic factor (GNDF), granulocyte colony stimulating factor (G-CSF), granulocyte-macrophage colony stimulating factor (GM-CSF), growth differentiation factor (GDF-9), hepatocyte growth factor (HGF), hepatoma derived growth factor (HDGF), insulin-like growth factor (IGF), migration-stimulating factor, myostatin (GDF-8), myelomonocytic growth factor (MGF), nerve growth factor (NGF), placental growth factor (P1GF), platelet-derived growth factor (PDGF), thrombopoietin (Tpo), transforming growth factor alpha (TGF-α), TGF-β, tumor necrosis factor alpha (TNF-α), vascular endothelial growth factor (VEGF), or a Wnt protein. In any of the above embodiments, a sufficient number of said FPUs to comprise 1×10⁶ cells produces at least 1.0 to 10 μM said cytokine in in vitro culture in growth medium over 24 hours.

In other more specific embodiments, said protein or polypeptide is a soluble receptor for AM, Ang, BMP, BDNF, EGF, Epo, FGF, GNDF, G-CSF, GM-CSF, GDF-9 , HGF, HDGF, IGF, migration-stimulating factor, GDF-8 , MGF, NGF, P1GF, PDGF, Tpo, TGF-α, TGF-β, TNF-α, VEGF, or a Wnt protein. In other specific embodiments, a sufficient number of said FPUs to comprise 1×10⁶ cells produces at least 1.0 to 10 μM of said soluble receptor in in vitro culture in growth medium over 24 hours.

In other specific embodiments, said protein or polypeptide is an interleukin or an active portion thereof. In various more specific embodiments, said interleukin is interleukin-1 alpha (IL-1α), IL-1β, IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, both IL-12 alpha and beta subunits, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23p19 subunit, IL-23 p40 subunit, IL-23 p19 subunit and IL-23 p40 subunit together, IL-24, IL-25, IL-26, IL-27B, IL-27-p28, IL-27B and IL-27-p28 together, IL-28A, IL-28B, IL-29, IL-30, IL-31, IL-32, IL-33, IL-34, IL-35, IL-36α, IL-36β, IL-36γ. In other more specific embodiments, a sufficient number of said FPUs to comprise 1×10⁶ cells produces at least 1.0 to 10 μM of said interleukin or active portion thereof in in vitro culture in growth medium over 24 hours. In certain more specific embodiments, said protein or polypeptide is a soluble receptor for IL-1α, IL-1β, IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23 p40 subunit, IL-24, IL-25, IL-26, IL-27B, IL-27-p28, IL-28A, IL-28B, IL-29, IL-30, IL-31, IL-32, IL-33, IL-34, IL-35, IL-36α, IL-36β, IL-36Δ. In a more specific embodiment, a sufficient number of said FPUs to comprise 1×10⁶ cells produces at least 1.0 to 10 μM of said soluble receptor in in vitro culture in growth medium over 24 hours.

In another more specific embodiment, said protein is an interferon (IFN). In specific embodiments, said interferon is IFN-α, IFN-β, IFN-γ, IFN-λ1, IFN-λ2, IFN-λ3, IFN-K, IFN-ε, IFN-κ, IFN-τ, IFN-δ, IFN-λ, or IFN-ω. In other specific embodiments, a sufficient number of said FPUs to comprise 1×10⁶ cells produces at least 1.0 to 10 μM of said interferon in in vitro culture in growth medium over 24 hours.

In other more specific embodiments, said protein or polypeptide is a soluble receptor for IFN-α, IFN-β, IFN-γ, IFN-λ1, IFN-λ2, IFN-λ3, IFN-K, IFN-ε, IFN-κ, IFN-τ, IFN-δ, IFN-λ, IFN-ω, of IFN-v. In certain specific embodiments, a sufficient number of said FPUs to comprise 1×10⁶ cells produces at least 1.0 to 10 μM of said soluble receptor in in vitro culture in growth medium over 24 hours.

In another specific embodiment, said protein is insulin or proinsulin. In a specific embodiment, a sufficient number of said FPUs to comprise 1×10⁶ cells produces at least 1.0 to 10 μM of said insulin in in vitro culture in growth medium over 24 hours. In another specific embodiment, said protein is a receptor for insulin. In certain more specific embodiments, said cells producing insulin or proinsulin have additionally been genetically engineered to produce one or more of prohormone convertase 1, prohormone convertase 2, or carboxypeptidase E.

In another specific embodiment, said protein is leptin (LEP). In another specific embodiment, a sufficient number of said FPUs to comprise 1×10⁶ cells produces at least 1.0 to 10 μM of said leptin in in vitro culture in growth medium over 24 hours.

In another specific embodiment, said protein is erythropoietin. In another specific embodiment, a sufficient number of said FPUs to comprise 1×10⁶ cells produces at least 1.0 to 10 μM of said erythropoietin in in vitro culture in growth medium over 24 hours. In another specific embodiment, said protein is thrombopoietin. In another specific embodiment, a sufficient number of said FPUs to comprise 1×10⁶ cells produces at least 1.0 to 10 μM of said thrombopoietin in in vitro culture in growth medium over 24 hours.

In another specific embodiment, said protein is tyrosine 3-monooxygenase. In certain specific embodiments, a sufficient number of said FPUs to comprise 1×10⁶ cells produces at least 1.0 to 10 μM of L-DOPA in in vitro culture in growth medium over 24 hours. In a more specific embodiment, said cells expressing said tyrosine 3-monoosygenase have been further engineered to express aromatic L-amino acid decarboxylase. In a more specific embodiment, a sufficient number of said FPUs to comprise 1×10⁶ cells produces at least 1.0 to 10 μM of dopamine in in vitro culture in growth medium over 24 hours.

In certain other specific embodiments, said protein is a hormone or prohormone. In various specific embodiments, said hormone is antimullerian hormone (AMH), adiponectin (Acrp30), adrenocorticotropic hormone (ACTH), angiotensin (AGT), angiotensinogen (AGT), antidiuretic hormone (ADH), vasopressin, atrial-natriuretic peptide (ANP), calcitonin (CT), cholecystokinin (CCK), corticotrophin-releasing hormone (CRH), erythropoietin (Epo), follicle-stimulating hormone (FSH), testosterone, estrogen, gastrin (GRP), ghrelin, glucagon (GCG), gonadotropin-releasing hormone (GnRH), growth hormone (GH), growth hormone releasing hormone (GHRH), human chorionic gonadotropin (hCG), human placental lactogen (HPL), inhibin, leutinizing hormone (LH), melanocyte stimulating hormone (MSH), orexin, oxytocin (OXT), parathyroid hormone (PTH), prolactin (PRL), relaxin (RLN), secretin (SCT), somatostatin (SRIF), thrombopoietin (Tpo), thyroid-stimulating hormone (Tsh), and/or thyrotropin-releasing hormone (TRH).

In another specific embodiment, protein is cytochrome P450 side chain cleavage enzyme (P450SCC).

In another specific embodiment, said protein is a protein missing or malfunctioning in an individual who has a genetic disorder or disease. In certain specific embodiments, said genetic disease is familial hypercholesterolemia and said protein is low density lipoprotein receptor (LDLR); said genetic disease is polycystic kidney disease, and said protein is polycystin-1 (PKD1), PKD-2 or PKD3; or said genetic disease is phenylketonuria and said protein is phenylalanine hydroxylase.

In a specific embodiment of any of the FPUs disclosed herein, said FPUs comprise an immune suppressive compound or an anti-inflammatory compound. In specific embodiments, said immune-suppressive or anti-inflammatory compound is a non-steroidal anti-inflammatory drug (NSAID), acetaminophen, naproxen, ibuprofen, acetylsalicylic acid, a steroid, an anti-T cell receptor antibody, an anti-IL-2 receptor antibody, basiliximab, daclizumab (ZENAPAX)®), anti T cell receptor antibodies (e.g., Muromonab-CD3), azathioprine, a corticosteroid, cyclosporine, tacrolimus, mycophenolate mofetil, sirolimus, calcineurin inhibitors, and the like. In a specific embodiment, the immumosuppressive agent is a neutralizing antibody to macrophage inflammatory protein (MIP)-1α or MIP-1β.

In certain embodiments of any of the FPUs disclosed herein, said FPUs dissolve or degrade within a recipient of the FPUs. In certain other embodiments of any of the FPUs disclosed herein, said FPUs maintain structural integrity, and/or substantially maintains cellular composition, within a recipient of the FPUs. In certain other embodiments of any of the FPUs disclosed herein, said FPUs maintain said at least one physiological function for 1, 2, 3, 4, 5, 6, or 7 days, or for 1, 2, 3, 4, 5, 6, 7, 8, 9, 10 or more weeks after administration to an individual.

In certain specific embodiments of any of the FPUs presented herein, said FPUs perform at least one function of a liver, kidney, pancreas, thyroid or lung.

The FPUs can comprise pituitary-specific cells, and/or cells that perform pituitary-specific functions. In certain embodiments, any of the FPUs presented herein comprises pituitary gland acidophil cells. In certain other embodiments, any of the FPUs presented herein comprises pituitary basophil cells. In certain other embodiments, any of the FPUs presented herein comprises both pituitary gland acidophil cells and basophil cells. In another embodiment, any of the FPUs presented herein comprises pituitary somatotropes. In another embodiment, any of the FPUs presented herein comprises pituitary mammotrophs. In another embodiment, any of the FPUs presented herein comprises pituitary corticotrophs. In another embodiment, any of the FPUs presented herein comprises pituitary thyrotrophs. In another embodiment, any of the FPUs presented herein comprises pituitary gonadotrophs. In another embodiment, any of the FPUs presented herein comprises said FPUs comprise two or more of pituitary somatotrophs, pituitary mammotrophs, pituitary corticotrophs, pituitary thyrotrophs, and/or pituitary gonadotrophs. In another embodiment of any of the FPUs presented herein, said FPUs produce a measurable amount of growth hormone (GH) in in vitro culture. In another embodiment of any of the FPUs presented herein, said FPUs produce a measurable amount of somatotrophic hormone (STH) in in vitro culture. In another embodiment of any of the FPUs presented herein, said FPUs produce a measurable amount of prolactin (PRL) in in vitro culture. In another embodiment of any of the FPUs presented herein, said FPUs produce a measurable amount of adrenocorticotropic hormone (ACTH) in in vitro culture. In another embodiment of any of the FPUs presented herein, said FPUs produce a measurable amount of melanocyte-stimulating hormone (MSH) in in vitro culture. In another embodiment of any of the FPUs presented herein, said FPUs produce a measurable amount of thyroid-stimulating hormone (TSH) in in vitro culture. In another embodiment of any of the FPUs presented herein, said FPUs produce a measurable amount of follicle-stimulating hormone (FSH) in in vitro culture. In another embodiment of any of the FPUs presented herein, said FPUs produce a measurable amount of leutinizing hormone (LH) in in vitro culture. In another embodiment of any of the FPUs presented herein, said FPUs comprise cells that produce one or more of GH, STH, PRL, ACTH, MSH, TSH, FSH, and/or LH. In a specific embodiment, said cells have been genetically engineered to produce one or more of GH, STH, PRL, ACTH, MSH, TSH, FSH, and/or LH.

In another embodiment of any of the FPUs presented herein, said FPUs comprise hypothalamic neurons and/or pituicytes. In another embodiment of any of the FPUs presented herein, said FPUs produce a measurable amount of antidiuretic hormone (ADH) in in vitro culture. In another embodiment of any of the FPUs presented herein, said FPUs produce a measurable amount of oxytocin in in vitro culture. In another embodiment of any of the FPUs presented herein, said FPUs comprise cells that produce one or both of ADH and/or oxytocin. In a specific embodiment, said FPUs comprise cells that have been genetically engineered to produce one or both of ADH and/or oxytocin.

In specific embodiments, any of the FPUs provided herein comprise endothelial vessel-forming cells. In other specific embodiments, said FPUs comprise a plurality of vessels, e.g., blood vessels and/or lymphatic vessels. In more specific embodiments, said plurality of vessels constitute a reticulated or anastomosing network of said vessels.

The FPUs can comprise thyroid gland-specific cells, and/or cells that perform thyroid gland-specific functions. In certain embodiments, any of the FPUs provided herein comprise thyroid epithelial cells. In certain embodiments, any of the FPUs provided herein comprise thyroid parafollicular cells. In certain embodiments, any of the FPUs provided herein comprise thyroglobulin-producing cells. In certain embodiments, any of the FPUs provided herein comprise two or more of thyroid epithelial cells, thyroid parafollicular cells, and thyroglobulin-producing cells. In specific embodiments, any of the FPUs provided herein comprise endothelial vessel-forming cells. In other specific embodiments, said FPUs comprise a plurality of vessels, e.g., blood vessels and/or lymphatic vessels. In certain embodiments of any of the FPUs presented herein, said FPUs produce a measurable amount of thyroxine (T4) in in vitro culture. In certain other embodiments of any of the FPUs presented herein, said FPUs produce a measurable amount of triiodothyronine (T3) in in vitro culture. In certain other embodiments of any of the FPUs presented herein, said FPUs produce a measurable amount of calcitonin. In certain other embodiments of any of the FPUs presented herein, said FPUs comprise cells that produce one or more of T3, T4 and/or calcitonin. In more specific embodiments, said FPUs comprise cells genetically engineered to produce one or more of T3, T4 and/or calcitonin.

The FPUs can also comprise parathyroid gland-specific cells, or cells that perform parathyroid-specific functions. In certain embodiments of any of the FPUs presented herein, said FPUs comprise parathyroid chief cells. In other embodiments of any of the FPUs presented herein, said FPUs comprise parathyroid oxyphil cells. In other embodiments of any of the FPUs presented herein, said FPUs comprise both parathyroid chef cells and parathyroid oxyphil cells. In certain embodiments, any of the FPUs provided herein comprise endothelial vessel-forming cells. In other specific embodiments, said FPUs comprise a plurality of vessels, e.g., blood vessels and/or lymphatic vessels. In more specific embodiments, said plurality of vessels constitutes a reticulated or anastomosing network of said vessels. In certain embodiments of any of the FPUs presented herein, said FPUs produce a measurable amount of parathyroid hormone (PTH) in in vitro culture. In other embodiments of any of the FPUs presented herein, said FPUs comprise cells that produce PTH. In more specific embodiments, said FPUs comprise cells that have been genetically engineered to produce said PTH.

The FPUs can comprise adrenal gland-specific cells, and/or cells that perform adrenal gland-specific functions. In certain embodiments of any of the FPUs presented herein, said FPUs comprise adrenal gland zona glomerulosa cells. In other embodiments of any of the FPUs presented herein, said FPUs comprise adrenal gland fasciculate cells. In other embodiments of any of the FPUs presented herein, said FPUs comprise adrenal gland zona reticulata cells. In other embodiments of any of the FPUs presented herein, said FPUs comprise adrenal gland chromaffin cells. In certain embodiments, any of the FPUs provided herein comprise endothelial vessel-forming cells. In other specific embodiments, said FPUs comprise a plurality of vessels, e.g., blood vessels and/or lymphatic vessels. In more specific embodiments, said plurality of vessels constitutes a reticulated or anastomosing network of said vessels. In certain embodiments of any of the FPUs presented herein, said FPUs produce a measurable amount of aldosterone in in vitro culture. In other embodiments of any of the FPUs presented herein, said FPUs produce a measurable amount of 18 hydroxy 11 deoxycorticosterone in in vitro culture. In other embodiments of any of the FPUs presented herein, said FPUs produce a measurable amount of fludrocortisone in in vitro culture. In other embodiments of any of the FPUs presented herein, said FPUs produce a measurable amount of cortisol. In other embodiments of any of the FPUs presented herein, said FPUs produce a measurable amount of a non-cortisol glucocorticoid. In other embodiments of any of the FPUs presented herein, said FPUs produce a measurable amount of epinephrine. In other embodiments of any of the FPUs presented herein, said FPUs produce a measurable amount of adrenosterone. In other embodiments of any of the FPUs presented herein, said FPUs produce a measurable amount of dehydroepiandreosterone. In other embodiments of any of the FPUs presented herein, said FPUs comprise cells that produce one or more of aldosterone, 18 hydroxy 11 deoxycorticosterone, cortisol, fludrocortisones, a non-cortisol glucocorticoid, epinephrine, adrenosterone, and/or dehydroepiandrosterone. In other embodiments of any of the FPUs presented herein, said FPUs produce two or more of aldosterone, 18 hydroxy 11 deoxycorticosterone, cortisol, fludrocortisones, a non-cortisol glucocorticoid, epinephrine, adrenosterone, and/or dehydroepiandrosterone. In more specific embodiments, said FPUs comprise cells that have been genetically engineered to produce one or more of aldosterone, 18 hydroxy 11 deoxycorticosterone, cortisol, fludrocortisones, a non-cortisol glucocorticoid, epinephrine, adrenosterone, and/or dehydroepiandrosterone.

The FPUs provided herein can comprise liver-specific cells, or cells that perform one or more liver-specific functions. In certain embodiments of any of the FPUs provided herein, said FPUs comprise hepatocytes. In various embodiments of any of the FPUs provided herein, said FPUs produce a measurable amount of one or more of coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin HA; blood coagulation factor XIV), protein S and/or antithrombin. In various other embodiments of any of the FPUs provided herein, said FPUs produce detectable amounts of glucose from an amino acid, lactate, glycerol or glycogen. In other embodiments, said FPUs produce detectable amounts of insulin-like growth factor (IGF-1) or thrombopoietin. In other embodiments, said FPUs produce bile. In certain embodiments of any of the FPUs provided herein, said FPUs comprise cells that produce one or more of coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin HA; blood coagulation factor XIV), protein S, antithrombin, IGF-1 or thrombopoietin. In certain embodiments of any of the FPUs provided herein, said FPUs comprise hepatic vessel endothelial cells. In a specific embodiment, said hepatic vessel endothelial cells are disposed within said FPUs so as to define one or more vessels. In a more specific embodiment, said hepatocytes are disposed along and substantially parallel to said vessels. In a more specific embodiment, a plurality of said vessels are disposed in substantially radial fashion so as to define an exterior and an interior of said FPU, such that each vessel has a distal and a proximal end. In another more specific embodiment, said FPUs comprise at least one vessel that connects each of said distal ends of said vessels.

The FPUs provided herein can also comprise pancreatic cells, or can comprise cells that perform at least one pancreatic cell-specific function. In certain embodiments, said pancreatic cells are pancreatic alpha cells. In certain embodiments of any of the FPUs provided herein, said FPUs comprise pancreatic beta cells. In other embodiments of any of the FPUs provided herein, said FPUs comprise pancreatic delta cells. In other embodiments of any of the FPUs provided herein, said FPUs comprise pancreatic PP cells. In other embodiments of any of the FPUs provided herein, said FPUs comprise pancreatic epsilon cells. In other embodiments of any of the FPUs provided herein, said FPUs comprise two or more of pancreatic alpha cells, pancreatic beta cells, pancreatic delta cells, pancreatic PP cells, and/or pancreatic epsilon cells. In other embodiments of any of the FPUs provided herein, said FPUs produce a detectable amount of glucagon. In other embodiments of any of the FPUs provided herein, said FPUs produce a detectable amount of insulin. In other embodiments of any of the FPUs provided herein, said FPUs produce a detectable amount of amylin. In a more specific embodiment, said FPUs produce a detectable amount of insulin and a detectable amount of amylin. In a more specific embodiment, said insulin and said amylin in a ratio of about 50:1 to about 200:1. In other embodiments of any of the FPUs provided herein, said FPUs produce a detectable amount of somatostatin. In other embodiments of any of the FPUs provided herein, said FPUs produce a detectable amount of grehlin. In other embodiments of any of the FPUs provided herein, said FPUs produce a detectable amount of pancreatic polypeptide. In other embodiments of any of the FPUs provided herein, said FPUs comprise cells that produce a detectable amount of one or more of insulin, glucagon, amylin, somatostatin, pancreatic polypeptide, and/or grehlin.

In another aspect, further provided herein are methods of making Functional Physiological Units (FPUs). In one embodiment, provided herein is a method of making a functional physiological unit (FPU), comprising combining an isolated extracellular matrix (ECM) and at least one type of cell, such that said FPUs perform at least one function of an organ or tissue from an organ, wherein said FPUs is less than about 1000 microliters in volume, and wherein said at least one function of an organ or tissue from an organ is production of a protein, cytokine, interleukin, or small molecule characteristic of at least one cell type from said organ or tissue. In specific embodiments, said FPUs are less than about 100 microliters in volume; less than about 1 microliter in volume; less than about 100 picoliters in volume; or less than about 10 picoliters in volume. In other specific embodiments, said FPUs are less than about 10 millimeters along its longest axis; less than about 1 millimeter along its longest axis; or less than about 100 μM along its longest axis. In other specific embodiments, said FPUs comprise no more than about 10⁵ cells; no more than about 10⁴ cells; no more than about 10³ cells; or no more than about 10² cells.

In certain embodiments, the method comprises combining said cells and said ECM so as to provide at least one channel that traverses said FPU, wherein said channel facilitates diffusion of nutrients and/or oxygen to said cells. In certain other embodiments, the method additionally comprises combining said cells and said ECM with a synthetic matrix. In a specific embodiment, the synthetic matrix stabilizes the three-dimensional structure of said FPU. In another specific embodiment, said synthetic matrix comprises a polymer or a thermoplastic. In a more specific embodiment, said synthetic matrix is a polymer or a thermoplastic. In more specific embodiments, said thermoplastic is polycaprolactone, polylactic acid, polybutylene terephthal ate, polyethylene terephthalate, polyethylene, polyester, polyvinyl acetate, or polyvinyl chloride. In other more specific embodiments, said polymer is polyvinylidine chloride, poly(o-carboxyphenoxy)-p-xylene) (poly(o-CPX)), poly(lactide-anhydride) (PLAA), n-isopropyl acrylamide, acrylamide, pent erythritol diacrylate, polymethyl acrylate, carboxymethylcellulose, or poly(lactic-co-glycolic acid) (PLGA). In another more specific embodiment, said polymer is polyacrylamide.

In specific embodiments of the method, said extracellular matrix is placental extracellular matrix, e.g., telopeptide placental collagen. In a more specific embodiment of the method, said extracellular matrix is placental extracellular matrix comprising base-treated and/or detergent treated Type I telopeptide placental collagen that has not been chemically modified or contacted with a protease, wherein said ECM comprises less than 5% fibronectin or less than 5% laminin by weight; between 25% and 92% Type I collagen by weight; between 2% and 50% Type III collagen; between 2% and 50% type IV collagen by weight; and/or less than 40% elastin by weight. In a more specific embodiment, said telopeptide placental collagen is base-treated, detergent treated Type I telopeptide placental collagen, wherein said collagen has not been chemically modified or contacted with a protease, and wherein said composition comprises less than 1% fibronectin by weight; less than 1% laminin by weight; between 74% and 92% Type I collagen by weight; between 4% and 6% Type III collagen by weight; between 2% and 15% type IV collagen by weight; and/or less than 12% elastin by weight.

In certain embodiments of the method, said FPUs have substantially the shape of a rectangular block, a cube, a sphere, a spheroid, a rod, a cylinder, or a torus. In other embodiments, said FPUs comprise voids, communicating with the surface of said FPUs, large enough to permit entry or exit of cells. In other embodiments, said FPUs comprises voids, communicating with the surface of said FPUs, not large enough to permit entry or exit of cells.

In certain embodiments of the method, said ECM is crosslinked or stabilized. In a specific embodiment, said ECM is combined with a polymer that stabilizes the three-dimensional structure of said FPU. In specific embodiments, said combining is performed by printing, e.g., bioprinting, said cells and aid ECM together. In a more specific embodiment, said printing uses inkjet printing technology.

In other embodiments, at least part of the surface of said FPUs are covered with an extracellular matrix or a polymer. In a more specific embodiment, substantially all of the surface of said FPUs are covered with an extracellular matrix or a polymer.

In one embodiment of the method, said combining is performed by adding cells to a hydrophilic solution comprising said ECM; forming a sphere by dropping said solution into a hydrophobic liquid; allowing the ECM in said sphere to harden; and collecting said spheres.

The method can comprise the construction of FPUs comprising cells from, or cells that perform at least one physiological function of, an organ, e.g., a gland. In certain specific embodiments of the method, for example, said at least one type of cells comprises pituitary gland acidophil cells. In other specific embodiments, said at least one type of cells comprises pituitary basophil cells. In other specific embodiments, said at least one type of cells comprises both pituitary gland acidophil cells and basophil cells. In another specific embodiment, said at least one type of cells comprises pituitary somatotrophs. In another specific embodiment of the method, said at least one type of cells comprises pituitary mammotrophs. In another specific embodiment, said at least one type of cells comprises pituitary corticotrophs. In another specific embodiment, said at least one type of cells comprises pituitary thyrotrophs. In another specific embodiment, said at least one type of cells comprises pituitary gonadotrophs. In another specific embodiment, said FPUs comprise two or more of pituitary somatotrophs, pituitary mammotrophs, pituitary corticotrophs, pituitary thyrotrophs, and/or pituitary gonadotrophs. In a specific embodiment of any of the above method embodiments, said at least one type of cells additionally comprises vascular endothelial cells. In a more specific embodiment, said vascular endothelial cells are disposed within said FPUs so as to form one or more vessels. In a more specific embodiment, any of said pituitary somatotrophs, pituitary mammotrophs, pituitary corticotrophs, pituitary thyrotrophs, and/or pituitary gonadotrophs are disposed along said vessels during said combining. In a specific embodiment of the method, said FPUs produce a measurable amount of growth hormone (GH) in in vitro culture. In another specific embodiment, said FPUs produce a measurable amount of somatotrophic hormone (STH) in in vitro culture. In another specific embodiment, said FPUs produce a measurable amount of prolactin (PRL) in in vitro culture. In another specific embodiment, said FPUs produce a measurable amount of adrenocorticotropic hormone (ACTH) in in vitro culture. In another specific embodiment, said FPUs produce a measurable amount of melanocyte-stimulating hormone (MSH) in in vitro culture. In another specific embodiment, said FPUs produce a measurable amount of thyroid-stimulating hormone (TSH) in in vitro culture. In another specific embodiment, said FPUs produce a measurable amount of follicle-stimulating hormone (FSH) in in vitro culture. In another specific embodiment, said FPUs produce a measurable amount of leutinizing hormone (LH) in in vitro culture. In another specific embodiment, said FPUs comprise cells that produce one or more of GH, STH, PRL, ACTH, MSH, TSH, FSH, and/or LH. In another specific embodiment, said FPUs comprise cells have been genetically engineered to produce one or more of GH, STH, PRL, ACTH, MSH, TSH, FSH, and/or LH. In another specific embodiment, said at least one type of cells comprises hypothalamic neurons. In another specific embodiment, said at least one type of cells comprises pituicytes. In a more specific embodiment, said at least one type of cells comprises both hypothalamic neurons and pituicytes. In a specific embodiment of the method, said FPUs produce a measurable amount of antidiuretic hormone (ADH) in in vitro culture. In another specific embodiment, said FPUs produce a measurable amount of oxytocin in in vitro culture. In a more specific embodiment of the method, said FPUs comprise cells that produce one or both of ADH and/or oxytocin. In certain specific embodiments, said FPUs comprise cells that have been genetically engineered to produce one or both of ADH and/or oxytocin. In certain specific embodiments of any of the above methods, said at least one type of cells additionally comprises endothelial vessel-forming cells. In a more specific embodiment, said endothelial vessel-forming cells are arranged during formation of said FPUs so as to produce a plurality of vessels in said FPUs. In a more specific embodiment, said endothelial vessel-forming cells are arranged during formation of said FPUs so as to produce a reticulated network of said vessels.

In certain other specific embodiments of the method, the FPUs perform at least one thyroid gland-specific function or parathyroid gland-specific function. In one specific embodiment, said at least one type of cells comprises thyroid epithelial cells. In another specific embodiment, said at least one type of cells comprises thyroid parafollicular cells. In another specific embodiment, said at least one type of cells comprises thyroglobulin-producing cells. In other specific embodiments, said at least one type of cells comprises two or more of thyroid epithelial cells, thyroid parafollicular cells, and thyroglobulin-producing cells. In another specific embodiment of the method, said at least one type of cells further comprises vascular endothelial cells. In another specific embodiment, said vascular endothelial cells are arranged, during production of said FPUs, so as to form one or more vessels, e.g., blood vessels and/or lymph vessels, in said FPUs. In another specific embodiment, said FPUs produce a measurable amount of thyroxine (T4) in in vitro culture. In another specific embodiment, said FPUs produce a measurable amount of triiodothyronine (T3) in in vitro culture. In another specific embodiment, said FPUs produce a measurable amount of calcitonin. In another specific embodiment, said one or more types of cells comprise cells that produce one or more of T3, T4 and/or calcitonin. In another specific embodiment of the method, said one or more types of cells comprises cells genetically engineered to produce one or more of T3, T4 and/or calcitonin. In another specific embodiment, said one or more types of cells comprises parathyroid chief cells. In another specific embodiment, said FPUs comprise parathyroid oxyphil cells. In a more specific embodiment, said FPUs comprise both parathyroid chef cells and parathyroid oxyphil cells. In another specific embodiment, said one or more types of cells comprises vascular endothelial cells. In a more specific embodiment, said vascular endothelial cells are arranged, during construction of said FPU, so as to form one or more vessels in said FPU. In a more specific embodiment, said FPUs comprise a plurality of vessels. In another specific embodiment, said FPUs produce a measurable amount of parathyroid hormone (PTH) in in vitro culture. In another specific embodiment, said FPUs comprise cells that produce PTH. In another specific embodiment, said one or more types of cells comprises cells that have been genetically engineered to produce said PTH.

In certain other specific embodiments of the method, the FPUs perform at least one adrenal gland-specific physiological function. In a specific embodiment, said one or more types of cells comprises adrenal gland zona glomerulosa cells. In another specific embodiment, said one or more types of cells comprises adrenal gland fasciculate cells. In another specific embodiment, said one or more types of cells comprises adrenal gland zona reticulata cells. In another specific embodiment, said one or more types of cells comprises adrenal gland chromaffin cells. In another specific embodiment, said one or more types of cells comprises vascular endothelial cells. In another specific embodiment, said vascular endothelial cells arc arranged, during construction of said FPU, so as to form one or more vessels in said FPU. In another specific embodiment, said FPUs produce a measurable amount of aldosterone in in vitro culture. In another specific embodiment, said FPUs produce a measurable amount of 18 hydroxy 11 deoxycorticosterone in in vitro culture. In another specific embodiment, said FPUs produce a measurable amount of fludrocortisone in in vitro culture. In another specific embodiment, said FPUs produce a measurable amount of cortisol. In another specific embodiment, said FPUs produce a measurable amount of a non-cortisol glucocorticoid. In another specific embodiment, said FPUs produce a measurable amount of epinephrine. In another specific embodiment, said FPUs produce a measurable amount of adrenosterone. In another specific embodiment, said FPUs produce a measurable amount of dehydroepiandrosterone. In another specific embodiment of the method, said one or more types of cells comprises cells that produce one or more of aldosterone, 18 hydroxy 11 deoxycorticosterone, cortisol, fludrocortisones, a non-cortisol glucocorticoid, epinephrine, adrenosterone, and/or dehydroepiandrosterone. In a more specific embodiment, said one or more types of cells comprises cells that have been genetically engineered to produce one or more of aldosterone, 18 hydroxy 11 deoxycorticosterone, cortisol, fludrocortisones, a non-cortisol glucocorticoid, epinephrine, adrenosterone, and/or dehydroepiandrosterone. In another specific embodiment, said one or more types of cells comprises endothelial progenitor cells. In another specific embodiment, said vascular endothelial cells are arranged, during construction of said FPU, so as to form one or more vessels in said FPU. In another specific embodiment, said FPUs comprise a plurality of vessels, e.g., blood vessels and/or lymphatic vessels.

In certain other specific embodiments of the method, the FPUs perform at least one liver-specific function. In a specific embodiment, said one or more types of cells comprises hepatocytes. In another specific embodiment, said FPUs produce a measurable amount of one or more of coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin IIA; blood coagulation factor XIV), protein S and/or antithrombin. In another specific embodiment, said FPUs produce detectable amounts of glucose from an amino acid, lactate, glycerol or glycogen. In another specific embodiment, said FPUs produce detectable amounts of insulin-like growth factor (IGF-1) or thrombopoietin. In another specific embodiment, said FPUs produce bile. In another specific embodiment, said FPUs comprise cells that produce one or more of coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin IIA; blood coagulation factor XIV), protein S, antithrombin, IGF-1 or thrombopoietin. In another specific embodiment of the method, said one or more types of cells additionally comprises hepatic vessel endothelial cells. In a more specific embodiment, said hepatic vessel endothelial cells are disposed within said FPUs so as to define one or more vessels. In a more specific embodiment, said hepatocytes are disposed along and substantially parallel to said vessels. In a more specific embodiment, a plurality of said vessels are disposed in substantially radial fashion so as to define an exterior and an interior of said FPU, such that each vessel has a distal and a proximal end. In another more specific embodiment, said FPUs comprise at least one vessel that connects each of said distal ends of said vessels.

In other specific embodiments of the method, said FPUs perform one or more functions of a pancreas. In a specific embodiment, said one or more types of cells comprises pancreatic alpha cells. In another specific embodiment, said one or more types of cells comprises pancreatic beta cells. In another specific embodiment, said one or more types of cells comprises pancreatic delta cells. In another specific embodiment, said one or more types of cells comprises pancreatic PP cells. In another specific embodiment, said one or more types of cells comprises pancreatic epsilon cells. In another specific embodiment, said FPUs comprise two or more of pancreatic alpha cells, pancreatic beta cells, pancreatic delta cells, pancreatic PP cells, and/or pancreatic epsilon cells. In certain specific embodiments, said FPUs produce a detectable amount of glucagon. In another specific embodiment, said FPUs produce a detectable amount of insulin. In another specific embodiment, said FPUs produce a detectable amount of amylin. In another specific embodiment, said FPUs produce a detectable amount of insulin and a detectable amount of amylin. In a more specific embodiment, said FPUs produce said insulin and said amylin in a ratio of about 50:1 to about 200:1. In another specific embodiment, said FPUs produce a detectable amount of somatostatin. In another specific embodiment, said FPUs produce a detectable amount of grehlin. In another specific embodiment, said FPUs produce a detectable amount of pancreatic polypeptide. In other specific embodiments, said FPUs comprise cells that produce a detectable amount of one or more of insulin, glucagon, amylin, somatostatin, pancreatic polypeptide, and/or grehlin.

In a specific embodiment, the FPUs described herein are not vascularized, e.g., do not comprise one or more blood vessels. In another specific embodiment, the FPUs described herein do not comprise cells (e.g., placental stem cells) derived or obtained from placenta, e.g., human placenta. In another specific embodiment, the FPUs described herein do not comprise tissue (e.g., extracellular matrix or components thereof) derived or obtained from placenta, e.g., human placenta.

In another aspect, provided herein are methods of using the Functional Physiological Units provided herein in methods of treating individuals, e.g., individuals suffering a deficiency in one or more biomolecules or physiological functions of an organ or tissue. In one embodiment, for example, provided herein is a method of treating an individual in need of human growth hormone (hGH) comprising administering to said individual a plurality of, e.g., a therapeutically effective amount of, FPUs producing hGH, or comprising cells that produce hGH. In certain other embodiments, provided herein is a method of treating an individual in need of somatotrophic hormone (STH) comprising administering to said individual a plurality of FPUs that produce, or which comprise cells that produce, STH.

In another embodiment, provided herein is a method of treating an individual in need of prolactin (PRL) comprising administering to said individual a plurality of, e.g., a therapeutically effective amount of, FPUs that produce, or which comprise cells that produce, PRL. In specific embodiment, said individual has one or more of metabolic syndrome, arteriogenic erectile dysfunction, premature ejaculation, oligozoospermia, asthenospermia, hypofunction of seminal vesicles, or hypoandrogenism.

In another embodiment, provided herein is a method of treating an individual in need of adrenocorticotropic hormone (ACTH) comprising administering to said individual a plurality of, e.g., a therapeutically effective amount of, FPUs producing, or comprising cells that produce, ACTH. In a specific embodiment, said individual has Addison's disease.

In another embodiment, provided herein is a method of treating an individual in need of melanocyte-stimulating hormone (MSH) comprising administering to said individual a plurality of, e.g., a therapeutically effective amount of, FPUs producing, or comprising cells that produce, MSH. In a specific embodiment, said individual has Alzheimer's disease.

In another embodiment, provided herein is a method of treating an individual in need of thyroid-stimulating hormone (TSH) comprising administering to said individual a plurality of, e.g., a therapeutically effective amount of, FPUs producing, or comprising cells that produce, TSH. In a specific embodiment, said individual has or manifests cretinism.

In another embodiment, provided herein is a method of treating an individual in need of follicle-stimulating hormone (FSH) comprising administering to said individual a plurality of, e.g., a therapeutically effective amount of, FPUs producing, or comprising cells that produce, FSH. In a specific embodiment, said individual has or manifests infertility or azoospermia.

In another embodiment, provided herein is method of treating an individual in need of leutenizing hormone (LH) comprising administering to said individual a plurality of, e.g., a therapeutically effective amount of, FPUs producing, or comprising cells that produce, LH. In a specific embodiment, said individual has or manifests low testosterone, low sperm count or infertility.

Further provided herein is a method of treating an individual in need of antidiuretic hormone (ADH) comprising administering to said individual a plurality of, e.g., a therapeutically effective amount of, FPUs producing, or comprising cells that produce, ADH. In a specific embodiment, said individual has hypothalamic diabetes insipidus.

In another embodiment, provided herein is a method of treating an individual in need of oxytocin comprising administering to said individual a plurality of, e.g., a therapeutically effective amount of, FPUs producing, or comprising cells that produce, oxytocin.

In another embodiment, provided herein is a method of treating an individual in need of thyroxine (T4) comprising administering to said individual a plurality of, e.g., a therapeutically effective amount of, FPUs producing, or comprising cells that produce, T4. In a specific embodiment, said individual has or manifests mental retardation, dwarfism, weakness, lethargy, cold intolerance, or moon face.

In another embodiment, provided herein is a method of treating an individual in need of triiodothyronine (T3) comprising administering to said individual a plurality of, e.g., a therapeutically effective amount of, FPUs producing, or comprising cells that produce, T3. In a specific embodiment, said individual has heart disease. In a more specific embodiment, said individual, prior to administration of said FPUs, has a serum concentration of T3 that is less than 3.1 pmol/L.

In another embodiment, provided herein is a method of treating an individual in need of calcitonin comprising administering to said individual a plurality of, e.g., a therapeutically effective amount of, FPUs producing, or comprising cells that produce, calcitonin. In a specific embodiment, said individual has osteoporosis or chronic autoimmune hypothyroidism.

Further provided herein is a method of treating an individual in need of parathyroid hormone (PTH) comprising administering to said individual a plurality of, e.g., a therapeutically effective amount of, FPUs producing, or comprising cells that produce, PTH.

In another embodiment, provided herein is a method of treating an individual in need of aldosterone comprising administering to said individual a plurality of, e.g., a therapeutically effective amount of, FPUs producing, or comprising cells that produce, aldosterone. In a specific embodiment, said individual has idiopathic hypoaldosteronism, hypereninemic hypoaldosteronism, or hyporeninemic hypoaldosteronism. In another specific embodiment, said individual has chronic renal insufficiency.

In another embodiment, provided herein is a method of treating an individual in need of 18 hydroxy 11 deoxycorticosterone comprising administering to said individual a plurality of, e.g., a therapeutically effective amount of, FPUs producing, or comprising cells that produce, 18 hydroxy 11 deoxycorticosterone.

Further provided herein is a method of treating an individual in need of fludrocortisone comprising administering to said individual a plurality of, e.g., a therapeutically effective amount of, FPUs producing, or comprising cells that produce, fludrocortisone.

In another embodiment, provided herein is a method of treating an individual in need of cortisol, the method comprising administering to said individual a plurality of, e.g., a therapeutically effective amount of, FPUs producing, or comprising cells that produce, cortisol. In a specific embodiment, said individual has acute adrenal deficiency, Addison's disease, or hypoglycemia.

In another embodiment, provided herein is a method of treating an individual in need of a non-cortisol glucocorticoid, the method comprising administering to said individual a plurality of, e.g., a therapeutically effective amount of, FPUs producing, or comprising cells producing, said non-cortisol glucocorticoid.

Further provided herein is a method of treating an individual in need of epinephrine, the method comprising administering to said individual a plurality of, e.g., a therapeutically effective amount of, FPUs producing, or comprising cells that produce, epinephrine.

In another embodiment, provided herein is a method of treating an individual in need of adrenosterone comprising administering to said individual a plurality of, e.g., a therapeutically effective amount of, FPUs producing, or comprising cells that produce, adrenosterone.

In another embodiment, provided herein is a method of treating an individual in need of dehydroepiandrosterone comprising administering to said individual a plurality of, e.g., a therapeutically effective amount of, FPUs producing, or comprising cells that produce, dehydroepiandrosterone.

In another embodiment, provided herein is a method of treating an individual in need of a compound, comprising administering a plurality of, e.g., a therapeutically effective amount of, FPUs producing said compound, wherein said compound is coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin HA; blood coagulation factor XIV), protein S and/or antithrombin.

In another embodiment, provided herein is a method of treating an individual in need of IGF-1 comprising administering to said individual a plurality of, e.g., a therapeutically effective amount of, FPUs producing, or comprising cells that produce, IGF-1.

In another embodiment, provided herein is a method of treating an individual in need of thrombopoietin comprising administering to said individual a plurality of, e.g., a therapeutically effective amount of, FPUs producing, or comprising cells that produce, thrombopoietin.

In another embodiment, provided herein is a method of treating an individual in need of glucagon comprising administering to said individual a plurality of, e.g., a therapeutically effective amount of, FPUs producing, or comprising cells that produce, glucagon.

In another embodiment, provided herein is a method of treating an individual in need of insulin comprising administering to said individual a plurality of, e.g., a therapeutically effective amount of, FPUs producing, or comprising cells that produce, insulin. In a specific embodiment, said individual has diabetes mellitus.

In another embodiment, provided herein is a method of treating an individual in need of amylin comprising administering to said individual a plurality of, e.g., a therapeutically effective amount of, FPUs producing, or comprising cells that produce, amylin.

In another embodiment, provided herein is a method of treating an individual in need of grehlin comprising administering to said individual a plurality of, e.g., a therapeutically effective amount of, FPUs producing, or comprising cells that produce, grehlin.

Further provided herein is a method of treating an individual in need of pancreatic polypeptide comprising administering to said individual a plurality of, e.g., a therapeutically effective amount of, FPUs producing, or comprising cells that produce, pancreatic polypeptide.

### 4 DETAILED DESCRIPTION OF THE INVENTION

### 4.1 Functional Physiological Units: Structure

The FPUs provided herein, in certain aspects, comprise in contiguous form an isolated extracellular matrix (ECM) and at least one type of cell, wherein said FPUs perform at least one function of an organ, or a tissue from an organ. In this context, the ECM is an ECM not produced by said at least one type of cell. Each organ, a physiological function of which can be substituted or augmented by said FPUs, has a particular cellular structure, e.g., arrangement of cells that makes up the organ. In certain embodiments, the FPUs provided herein wholly or partially recapitulate the structure of such an organ, e.g., with respect to two or more, or all, of the cell types present in the organ. In certain other embodiments, the FPUs provided herein comprise none of the cell types natively present in an organ, a function of which is to be replaced by the FPU; however, the FPUs comprise one or more cell types that perform the physiological function that is to be replaced. In specific embodiments, said at least one function of an organ, or tissue from an organ, is production of a protein, growth factor, cytokine, interleukin, or small molecule characteristic of at least one cell type from said organ or tissue.

The FPUs provided herein, in certain embodiments, are constructed to be implantable or administrable, e.g., by implantation, injection, intravenous infusion, or the like. The FPUs in certain embodiments are coated with one or more physiologically-acceptable compositions, e.g., a polysaccharide, hydrogel, synthetic polymer, or the like. Generally, FPUs can have the structure of a rectangular block, cube, sphere, spheroid, rod, cylinder, or torus, or may have no definable (e.g. geometric) shape. The FPUs may comprise voids, communicating with the surface of said FPU, that are large enough to peimit entry or exit of cells. The FPUs may comprise voids, communicating with the surface of said FPU, that are not large enough to permit entry or exit of cells.

In certain embodiments, said FPUs are less than about 1000 microliters in volume, less than about 100 microliters in volume; less than about 1 microliter in volume; less than about 100 picoliters in volume; or less than about 10 picoliters in volume. In other various embodiments, said FPUs are less than about 10 millimeters wide, e.g., along the longest axis; less than about 1 millimeter wide, e.g., along the longest axis; or less than about 100 micrometer wide, e.g., along the longest axis. In other specific embodiments, said FPUs comprise no more than about 10⁷ cells; no more than about 10⁶ cells; no more than about 10⁵ cells; no more than about 10⁴ cells; no more than about 10³ cells; or no more than about 10² cells.

The Functional Physiological Units provided herein are, in certain embodiments, self-contained and not dependent upon any extraneous substrate or support for function.

The FPUs provided herein, in certain embodiments, are constructed so as to facilitate administration to an individual by a medically-acceptable method or route of administration. For example, FPUs may be constructed of a size that facilitates administration by intravenous, intra-arterial, intrathecal, or intraspinal injection or infusion. FPUs may in other embodiments, be constructed of a size that facilitates surgical implantation into a tissue, or a bone, of an individual.

In certain embodiments, the FPUs are coated with a natural or artificial polymer, such as a hydrogel, collagen glue, fibrin glue, polyethylene, and/or polypropylene. Preferably, the coating is in the form of a microfine mesh that at least allows diffusion of nutrients, oxygen, and the like to at least some, or all, of the cells within the FPUs (whether or not the FPUs comprise one or more vessels).

In some embodiments, the cells/compositions are formulated to provide an encapsulated form, e.g., as described in, for example, U.S. Pat. No. 6,783,964. For example, the cells may be encapsulated in a microcapsule of from 50 or 100 micrometers to 1 or 2 mm in diameter that includes an internal cell-containing core of polysaccharide gum surrounded by a semipermeable membrane; a microcapsule that includes alginate in combination with polylysine, polyornithine, and combinations thereof. Other suitable encapsulating materials include, but are not limited to, those described in U.S. Pat. No. 5,702,444.

In certain embodiments, the FPUs are produced as layers of cells, e.g., a single cell thick, separated by a natural or artificial polymer, e.g., any of the natural or artificial polymers specified herein. In certain embodiments, the cells in said single cell-thick layer of cells arc arranged so as to form channels between said cells that, e.g., allow the passage of a fluid. Such fluid may contain, e.g., oxygen and/or nutrients, and may be large enough to pass erythrocytes without clogging. Such channels may be constructed of the polymer itself, or may be defined by vascular endothelial cells. In embodiments in which the FPUs comprise more than one of such single cell-thick layer, such FPUs may comprise 2, 3, 4, 5, 6, 7, 8, 9, 10 or more such layers. In effect, in such embodiments, an individual FPU constitutes a “chip” that may be handled, e.g., with a microprobe, tweezers, or the like. The exterior of such a chip may be coated in plastic or other protective material.

In certain embodiments, the FPUs are constructed so as to be used externally; that is, the FPUs, in certain embodiments, are connected to an individual by some physical connection (e.g., tubing) rather than being implanted directly into the individual. The “chip” described above may be so constructed to be used externally, and to be connected to an individual. In other embodiments, the FPUs are contained within a bioreactor, and the products of the FPUs are communicated to an individual by physical means, e.g., tubing that connects the bioreactor to the individual.

### 4.1.1 Extracellular Matrix

In certain embodiments, the FPUs provided herein comprise extracellular matrix. Said extracellular matrix (ECM) may contact, e.g., surround, some, or all, cells in said FPUs. In certain embodiments, said ECM is plant ECM (e.g., soybean ECM), mammalian ECM, piscene ECM, or molluscan ECM. In a specific embodiment, said ECM is or comprises placental telopeptide collagen. In another specific embodiment, said ECM is or comprises placental atelopeptide collagen. In a more specific embodiment, said ECM is the placental telopeptide collagen described in Bhatia, US 20080181935, the disclosure of which is hereby incorporated by reference in its entirety. In a more specific embodiment, said ECM is human placental ECM comprising base-treated and/or detergent treated Type I telopeptide placental collagen that has not been chemically modified or contacted with a protease, wherein said ECM comprises less than 5% fibronectin; less than 5% laminin by weight; between 25% and 92% Type I collagen by weight; less than about 40% elastin by weight; and 2% to 50% Type III collagen or 2% to 50% type IV collagen by weight. In a more specific embodiment, said extracellular matrix is placental extracellular matrix comprising base-treated, detergent treated Type I telopeptide placental collagen that has not been chemically modified or contacted with a protease, wherein said ECM comprises less than 1% fibronectin; less than 1% laminin by weight; between 74% and 92% Type I collagen by weight; less than about 12% elastin by weight; and 4% to 6% Type III collagen or 2% to 15% type IV collagen by weight.

In certain specific embodiments, said ECM, e.g., said telopeptide collagen, is derivatized prior to production of said FPUs, e.g., with a cell attachment peptide, a cell attachment protein, a cytokinc, or a glycosaminoglycan. Where dcrivatization is with a cytokinc, the cytokinc can be, e.g., vascular endothelial growth factor (VEGF), or a bone morphogenetic protein (BMP). In certain specific embodiments, said cell attachment peptide is a peptide comprising one or more RGD motifs, one or more RFYVVMWK motifs, one or more IRVVM motifs, and/or one or more RADS motifs, where the letters in said motifs are one-letter codes for amino acids. In certain other embodiments, the ECM may be derivatized with a peptide that inhibits cell attachment, e.g., a peptide having one or more RFYVVM motifs.

Placental ECM, e.g., comprising placental telopeptide collagen, useful in the preparation of the FPUs provided herein, may be prepared as follows. Such ECM is produced without having been chemically modified or contacted with a protease. First, placental tissue (either whole placenta or part thereof) is obtained by standard methods, e.g., collection as soon as practical after Caesarian section or normal birth, e.g., aseptically. The placental tissue can be from any part of the placenta including the amnion, whether soluble or insoluble or both, the chorion, the umbilical cord or from the entire placenta. In certain embodiments, the collagen composition is prepared from whole human placenta without the umbilical cord. The placenta may be stored at room temperature, or at a temperature of about 2° C. to 8° C., until further treatment. The placenta is preferably exsanguinated, i.e., completely drained of the placental and cord blood remaining after birth. The expectant mother, in certain embodiments, is screened prior to the time of birth, for, e.g., HIV, HBV, HCV, HTLV, syphilis, CMV, and other viral pathogens known to contaminate placental tissue.

The placental tissue may be decellularized prior to production of the telopeptide ECM. The placental tissue can be decellularized according to any technique known to those of skill in the art such as those described in detail in U.S. Patent Application Publication Nos. 20040048796 and 20030187515, the contents of which are hereby incorporated by reference in their entireties.

In a first step of preparing the ECM, the placental tissue is subjected to an osmotic shock. The osmotic shock can be in addition to any clarification step or it can be the sole clarification step according to the judgment of one of skill in the art. The osmotic shock can be carried out in any osmotic shock conditions known to those of skill in the art. Such conditions include incubating the tissue in solutions of high osmotic potential, or of low osmotic potential or of alternating high and low osmotic potential. The high osmotic potential solution can be any high osmotic potential solution known to those of skill in the art such as a solution comprising one or more of NaCl (e.g., 0.2-1.0 M), KCl (e.g., 0.2-1.0 or 2.0 M), ammonium sulfate, a monosaccharide, a disaccharide (e.g., 20% sucrose), a hydrophilic polymer (e.g., polyethylene glycol), glycerol, etc. In certain embodiments, the high osmotic potential solution is a sodium chloride solution, e.g., at least 0.25M, 0.5M, 0.75M, 11.0M, 1.25M, 1.5M, 1.75M, 2M, or 2.5M NaCl. In some embodiments, the sodium chloride solution is about 0.25-5M, about 0.5-4M, about 0.75-3M, or about 1.0-2.0M NaCl. The low osmotic potential solution can be any low osmotic potential solution known to those of skill in the art, such as water, for example water deionized according to any method known to those of skill. In some embodiments, the osmotic shock solution comprises water with an osmotic shock potential less than that of 50 mM NaCl.

In certain embodiments, the osmotic shock is in a sodium chloride solution followed by a water solution. In certain embodiments, one or two NaCl solution treatments are followed by a water wash.

The collagen composition resulting from the osmotic shock is then incubated with a detergent. The detergent can be any detergent known to those of skill in the art to be capable of disrupting cellular or subcellular membranes, e.g., an ionic detergent, a nonionic detergent, deoxycholate, sodium dodecylsulfate, Triton X 100, TWEEN®, or the like. Detergent treatment can be carried out at about 0° C. to about 30° C., about 5° C. to about 25° C., about 5° C. to about 20° C., about 5° C. to about 15° C., about 0° C., about 5° C., about 10° C., about 15° C., about 20° C., about 25° C., or about 30° C. Detergent treatment can be carried out for, e.g., about 1-24 hours, about 2-20 hours, about 5-15 hours, about 8-12 hours, or about 2-5 hours.

The collagen composition resulting from the detergent treatment is then incubated in basic conditions. Particular bases for the basic treatment include biocompatible bases, volatile bases, or any organic or inorganic bases at a concentration of, for example, 0.2-1.0M. In certain embodiments, the base is selected from the group consisting of NH₄OH, KOH and NaOH, e.g., 0.1M NaOH, 0.25M NaOH, 0.5M NaOH, or 1M NaOH. The base treatment can be carried out at, e.g., 0° C. to 30° C., 5° C. to 25° C., 5° C. to 20° C., 5° C. to 15° C., about 0° C., about 5° C., about 10° C., about 15° C., about 20° C., about 25° C., or about 30° C., for, e.g., about 1-24 hours, about 2-20 hours, about 5-15 hours, about 8-12 hours, or about 2-5 hours.

The ECM can be produced without treatment by a base; omission of a base treatment step typically results in a collagen composition comprising relatively higher amounts of elastin, fibronectin and/or laminin than the collagen composition produced with inclusion of the basic treatment.

Typically, the process described above for human placental tissue results in production of placental ECM comprising base-treated and/or detergent treated Type I telopeptide placental collagen that has not been chemically modified or contacted with a protease, wherein said ECM comprises less than 5% fibronectin or less than 5% laminin by weight; between 25% and 92% Type I collagen by weight; between 2% and 50% Type III collagen; between 2% and 50% type IV collagen by weight; and/or less than 40% elastin by weight. In a more specific embodiment, the process results in production of base-treated, detergent treated Type I telopeptide placental collagen, wherein said collagen has not been chemically modified or contacted with a protease, and wherein said composition comprises less than 1% fibronectin by weight; less than 1% laminin by weight; between 74% and 92% Type I collagen by weight; between 4% and 6% Type III collagen by weight; between 2% and 15% type IV collagen by weight; and/or less than 12% elastin by weight. In specific embodiments of any of the FPUs described herein, the FPUs comprise the base-treated, detergent-treated telopeptide collagen described above.

### 4.1.2 Synthetic Matrices

In addition to ECM, the FPUs provided herein may comprise one or more synthetic matrices, e.g., to provide improved structural integrity over the ECM+cells alone, to facilitate manufacture of the FPUs, or for any other compatible purpose. In a specific embodiment, said synthetic matrix stabilizes the three-dimensional structure of said FPU. In specific embodiments, said synthetic matrix is, or comprises, a polymer or a thermoplastic. Various polymers or thermoplastics, preferably biocompatible, may be used to construct said FPUs. For example, in various embodiments, said thermoplastic one or more of is polycaprolactone, polylactic acid, polybutylene terephthalate, polyethylene terephthalate, polyethylene, polyester, polyvinyl acetate, or polyvinyl chloride. In certain other specific embodiments, said polymer is polyvinylidine chloride, poly(o-carboxyphenoxy)-p-xylene) (poly(o-CPX)), poly(lactide-anhydride) (PLAA), n-isopropyl acrylamide, acrylamide, pent erythritol diacrylate, polymethyl acrylate, carboxymethylcellulose, or poly(lactic-co-glycolic acid) (PLGA). In certain other specific embodiments, said polymer is polyacrylamide.

### 4.2 Cells

Depending on the physiological function(s) the FPUs are designed to augment, or replace, the FPUs provided herein can comprise one or more relevant cell types.

In certain embodiments of any of the FPUs provided herein, for example, the one or more types of cells comprise cells of the immune system, e.g., T cells, B cells, dendritic cells, and/or natural killer (NK) cells. In a specific embodiment, said NK cells comprise, or are, CD56⁺ CD16⁻ placental intermediate natural killer (PiNK) cells, e.g., the placental NK cells described in US 2009/0252710, the disclosure of which is hereby incorporated by reference in its entirety.

In certain other embodiments of any of the FPUs provided herein, the one or more types of cells are, or comprise, isolated stem cells or progenitor cells. In specific embodiments, said isolated stem cells or progenitor cells are isolated embryonic stem cells, embryonic germ cells, induced pluripotent stem cells, mesenchymal stem cells, bone marrow-derived mesenchymal stem cells, bone marrow-derived mesenchymal stromal cells, tissue plastic-adherent placental stem cells (PDACs®), umbilical cord stem cells, amniotic fluid stem cells, amnion derived adherent cells (AMDACs) (e.g., as described in U.S. 2010/0124569), osteogenic placental adherent cells (OPACs) (e.g., as described in US 20100047214), adipose stem cells, limbal stem cells, dental pulp stem cells, myoblasts, endothelial progenitor cells, neuronal stem cells, exfoliated teeth derived stem cells, hair follicle stem cells, dermal stem cells, parthenogenically derived stem cells, reprogrammed stem cells, amnion derived adherent cells, or side population stem cells. In other specific embodiments, the one or more types of cells comprised within the FPUs are, or comprise, isolated hematopoietic stem cells or hematopoietic progenitor cells. In other specific embodiments, the one or more types of cells comprised within the FPUs are tissue culture plastic-adherent CD34⁻, CD10⁺, CD105⁺, and CD200⁺ placental stem cells, e.g., the placental stem cells described in U.S. Pat. Nos. 7,468,276 and 8,057,788, the disclosures of which are hereby incorporated by reference in their entireties. In a specific embodiment, said placental stem cells arc additionally one or more of CD45⁻, CD80⁻, CD86⁻, or CD90⁺. In a more specific embodiment, said placental stem cells are additionally CD45⁻, CD80⁻, CD86⁻, and CD90⁺.

Such placental stem cells are immunomodulatory. See, e.g., U.S. Pat. No. 7,682,803 and US 2008/0226595, the disclosures of which are hereby incorporated by reference in their entireties. In another specific embodiment, therefore, said placental stem cells, or said FPUs comprising said placental stem cells, when said FPUs are implanted into a recipient, suppress an immune response in said recipient. In another specific embodiment, any of said isolated stem cells recited above, or said FPUs comprising said isolated stem cells, wherein said isolated stem cells are immunomodulatory, suppress an immune response in a recipient when said FPUs are implanted into said recipient. In a specific embodiment, said FPUs, or the immunomodulatory stem cells comprised therein, suppress an immune response locally within said recipient, e.g., at or adjacent to a site of administration or implantation. In another specific embodiment, said FPUs, or the immunomodulatory stem cells comprised therein, suppress an immune response globally within said recipient.

In various other specific embodiments, the FPUs comprise one or more cell types, wherein said one or more cell types are, or comprise, differentiated cells, e.g., one or more of endothelial cells, epithelial cells, derrnal cells, endodermal cells, mesodermal cells, fibroblasts, osteocytes, chondrocytes, natural killer cells, dendritic cells, hepatic cells, pancreatic cells, or stromal cells. In various more specific embodiments, said differentiated cells are, or comprise salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells, cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus cffcrcns nonciliatcd cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

In specific embodiments of any of the FPUs comprising any of the cell types listed herein, the at least one type of cells are primary culture cells, cells that have been directly obtained from a tissue or organ without culturing, cells that have been cultured in vitro, or cells of a cell line, e.g., partially, conditionally, or fully immortalized cells.

### 4.3 Physiological Functions Replicated By The FPUs

A primary function of the FPUs provided herein is that the FPUs, by the cells comprised within them, perform a physiological function. More specifically, the FPUs and/or the cells comprised within them replicate or augment one or more physiological functions of an organ or a tissue in an individual who is a recipient of said FPUs. In certain embodiments, as above, the FPUs comprise isolated primary or cultured cells that perform the one or more physiological functions. In other embodiments, the FPUs comprise cells have been genetically engineered to perform the physiological function. In a specific embodiment, said genetically engineered cells produce a protein or polypeptide not naturally produced by the corresponding un-engineered cells, or have been genetically engineered to produce a protein or polypeptide in an amount greater than that naturally produced by the corresponding un-engineered cells, wherein said cellular composition comprises differentiated cells.

In embodiments in which the physiological function is production of a protein or polypeptide, in specific embodiments, said protein or polypeptide is a cytokine or a peptide comprising an active part thereof. In more specific embodiments, said cytokine is adrenomedullin (AM), angiopoietin (Ang), bone morphogenetic protein (BMP), brain-derived neurotrophic factor (BDNF), epidermal growth factor (EGF), erythropoietin (Epo), fibroblast growth factor (FGF), glial cell line-derived neurotrophic factor (GNDF), granulocyte colony stimulating factor (G-CSF), granulocyte-macrophage colony stimulating factor (GM-CSF), growth differentiation factor (GDF-9), hepatocyte growth factor (HGF), hepatoma derived growth factor (HDGF), insulin-like growth factor (IGF), migration-stimulating factor, myostatin (GDF-8), myelomonocytic growth factor (MGF), nerve growth factor (NGF), placental growth factor (PIGF), platelet-derived growth factor (PDGF), thrombopoietin (Tpo), transforming growth factor alpha (TGF-α), TGF-β, tumor necrosis factor alpha (TNF-α), vascular endothelial growth factor (VEGF), or a Wnt protein. In a more specific embodiment of said FPUs, a sufficient number of said FPUs to comprise 1×10⁶ cells produces at least 1.0 to 10 μM said cytokine in in vitro culture in growth medium over 24 hours.

In other specific embodiments, said protein or polypeptide is a soluble receptor for AM, Ang, BMP, BDNF, EGF, Epo, FGF, GNDF, G-CSF, GM-CSF, GDF-9 , HGF, HDGF, IGF, migration-stimulating factor, GDF-8 , MGF, NGF, PlGF, PDGF, Tpo, TGF-α, TGF-β, TNF-α, VEGF, or a Wnt protein. In a more specific embodiment of said FPUs, a sufficient number of said FPUs to comprise 1×10⁶ cells produces at least 1.0 to 10 μM of said soluble receptor in in vitro culture in growth medium over 24 hours.

In other specific embodiments, said protein or polypeptide is an interleukin, e.g., interleukin-1 alpha (IL-1α), IL-1β, IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, both IL-12 alpha and beta subunits, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23 p40 subunit, IL-23 p19 subunit and IL-23 p40 subunit together, IL-24, IL-25, IL-26, IL-27B, IL-27-p28, IL-27B and IL-27-p28 together, IL-28A, IL-28B, IL-29, IL-30, IL-31, IL-32, IL-33, IL-34, IL-35, IL-36α, IL-36β, IL-36γ. IN a more specific embodiment of said FPUs, a sufficient number of said FPUs to comprise 1×10⁶ cells produces at least 1.0 to 10 μM of said interleukin in in vitro culture in growth medium over 24 hours.

In other specific embodiments, said protein or polypeptide is a soluble receptor for IL-1α, IL-1β, IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23 p40 subunit, IL-24, IL-25, IL-26, IL-27B, IL-27-p28, IL-28A, IL-28B, IL-29, IL-30, IL-31, IL-32, IL-33, IL-34, IL-35, IL-36α, IL-36β, IL-36γ. In a more specific embodiment of said FPUs, a sufficient number of said FPUs to comprise 1×10⁶ cells produces at least 1.0 to 10 μM of said soluble receptor in in vitro culture in growth medium over 24 hours.

In other specific embodiments, said protein or polypeptide is an interferon (IFN), e.g.,IFN-α, IFN-β, IFN-γ, IFN-λ1, IFN-λ3, IFN-K, IFN-ε, IFN-κ, IFN-δ, IFN-λ, IFN-ω, or IFN-v. In a more specific embodiment, a sufficient number of said FPUs to comprise 1×10⁶ cells produces at least 1.0 to 10 μM of said interferon in in vitro culture in growth medium over 24 hours.

In other specific embodiments, said protein or polypeptide is a soluble receptor for IFN-α, IFN-β, IFN-γ, IFN-λ1, IFN-λ2, IFN-λ3, IFN-K, IFN-ε, IFN-κ, IFN-τ, IFN-δIFN-λ, IFNω, or IFN-v. In a more specific embodiment, a sufficient number of said FPUs to comprise 1×10⁶ cells produces at least 1.0 to 10 μM of said soluble receptor in in vitro culture in growth medium over 24 hours.

In other specific embodiments, said protein or polypeptide is insulin or proinsulin. In a more specific embodiment, a sufficient number of said FPUs to comprise 1×10⁶ cells produces at least 1.0 to 10 μM of said insulin in in vitro culture in growth medium over 24 hours. In other specific embodiments, said protein is a receptor for insulin. In a more specific embodiment, said cells have additionally been genetically engineered to produce one or more of prohormone convertase 1, prohormone convertase 2, or carboxypeptidase E.

In another specific embodiment, said protein or polypeptide is leptin (LEP). In a more specific embodiment, a sufficient number of said FPUs to comprise 1×10⁶ cells produces at least 1.0 to 10 μM of said leptin in in vitro culture in growth medium over 24 hours.

In other specific embodiments, said protein is erythropoietin (Epo). In a more specific embodiment, a sufficient number of said FPUs to comprise 1×10⁶ cells produces at least 1.0 to 10 μM of said Epo in in vitro culture in growth medium over 24 hours.

In another specific embodiment, said protein is thrombopoietin (Tpo). In a more specific embodiment, a sufficient number of said FPUs to comprise 1×10⁶ cells produces at least 1.0 to 10 μM of said Tpo in in vitro culture in growth medium over 24 hours.

The FPUs may be constructed to as to produce dopamine, or a precursor to dopamine. In a specific embodiment of any of the FPUs provided herein, for example, said protein is tyrosine 3-monooxygenase. In a more specific embodiment, a sufficient number of said FPUs to comprise 1×10⁶ cells produces at least 1.0 to 10 μM of L-DOPA in in vitro culture in growth medium over 24 hours. In a more specific embodiment, said cells are further engineered to express aromatic L-amino acid decarboxylase. In a more specific embodiment, a sufficient number of said FPUs to comprise 1×10⁶ cells produces at least 1.0 to 10 μM of dopamine in in vitro culture in growth medium over 24 hours.

In another specific embodiment, said protein or polypeptide is a hormone or prohormone. In more specific embodiments, said hormone is antimullerian hormone (AMH), adiponectin (Acrp30), adrenocorticotropic hormone (ACTH), angiotensin (AGT), angiotensinogen (AGT), antidiuretic hormone (ADH), vasopressin, atrial-natriuretic peptide (ANP), calcitonin (CT), cholecystokinin (CCK), corticotrophin-releasing hormone (CRH), erythropoietin (Epo), follicle-stimulating hormone (FSH), testosterone, estrogen, gastrin (GRP), ghrelin, glucagon (GCG), gonadotropin-releasing hormone (GnRH), growth hormone (GH), growth hormone releasing hormone (GHRH), human chorionic gonadotropin (hCG), human placental lactogen (HPL), inhibin, leutinizing hormone (LH), melanocyte stimulating hormone (MSH), orexin, oxytocin (OXT), parathyroid hormone (PTH), prolactin (PRL), relaxin (RLN), secretin (SCT), somatostatin (SRIF), thrombopoietin (Tpo), thyroid-stimulating hormone (Tsh), and/or thyrotropin-releasing hormone (TRH).

In another specific embodiment, said protein or polypeptide is cytochrome P450 side chain cleavage enzyme (P450SCC).

In other specific embodiments, said protein is a protein missing or malfunctioning in an individual who has a genetic disorder or disease. In specific embodiments, said genetic disease is familial hypercholesterolemia and said protein is low density lipoprotein receptor (LDLR); said genetic disease is polycystic kidney disease, and said protein is polycystin-1 (PKDI), PKD-2 or PKD3; or said genetic disease is phenylketonuria and said protein is phenylalanine hydroxylase.

In embodiments, in which the FPUs comprise immunomodulatory cells, as described elsewhere herein, the FPUs can further comprise one or more immunomodulatory compounds, e.g., compound is a non-steroidal anti-inflammatory drug (NSAID), acetaminophen, naproxen, ibuprofen, acetylsalicylic acid, a steroid, an anti-T cell receptor antibody, an anti-IL-2 receptor antibody, basiliximab, daclizumab (ZENAPAX)®), anti T cell receptor antibodies (e.g., Muromonab-CD3), azathioprine, a corticosteroid, cyclosporine, tacrolimus, mycophenolate mofetil, sirolimus, calcineurin inhibitors, and the like. In a specific embodiment, the immumosuppressive agent is a neutralizing antibody to macrophage inflammatory protein (MIP)-1α or MIP-1β.

### 4.4 Specific Examples of FPUs

Specific embodiments of gland-specific FPUs are provided below in each of Sections 4.4.1 to 4.4.6, below.

### 4.4.1 Pituitary Gland

The pituitary gland comprises a body of cells, acidophils and chromophils in the anterior pituitary and neurosecretory cells in the posterior pituitary, surrounded by an anastomosing network of blood vessels. In certain embodiments, therefore, provided herein are FPUs that performs at least one physiological function of a pituitary gland, e.g., provided herein are pituitary FPUs. In specific embodiments, said at least one physiological function of a pituitary gland is production of, or said pituitary FPUs produce, detectable amounts of one or more pituitary-specific hormones, e.g., one or more of human growth hormone (hGH), prolactin (PRL), adrenocorticotropic hormone (ACTH) (also referred to as corticotrophin), melanocyte-stimulating hormone (MSH), thyroid-stimulating hormone (TSH) (also referred to as thyrotrophin), follicle-stimulating hormone (FSH), leutenizing hormone (LH), antidiuretic hormone (ADH), and/or oxytocin. In certain embodiments, said FPUs comprise (e.g., additionally comprises), cells that have been genetically engineered to produce detectable amounts of one or more pituitary-specific hormones, e.g., one or more of human growth hormone (hGH), prolactin (PRL), adrenocorticotropic hormone (ACTH) (also referred to as corticotrophin), melanocyte-stimulating hormone (MSH), thyroid-stimulating hormone (TSH) (also referred to as thyrotrophin), follicle-stimulating hormone (FSH), leutenizing hormone (LH), antidiuretic hormone (ADH), and/or oxytocin.

Production of said one or more pituitary-specific hormones by said FPUs may be assayed, e.g., by commercially-available kits and assays. For example, hGH production may be assayed in vitro using the Human GH ELISA kit (AbFrontier Co., Ltd.; Seoul, KR); ACTH production may be assayed in vitro using the ACTH (1-39) EIA Kit (Bachem, Torrance, Calif.); MSH production may be assayed in vitro by the Human/Mouse/Rat MSH EIA Kit (Raybiotech, Inc.; Norcross Ga.); TSH production may be assayed in vitro using the Human TSH ELISA Kit (Calbiotech, Inc., Spring Valley, Calif.); FSH production can be assayed in vitro using the Human FSH ELISA Kit (Anogen, Mississauga, Ontario, Canada); LH production can be assayed in vitro using the ELISA Kit for Leutenizing Hormone (Uscn Life Science, Wuhan, China); ADH production may be assessed in vitro using the CLIA Kit for Antidiuretic Hormone (ADH) (Uscn Life Science, Wuhan, China); prolactin production by said FPUs can be assessed in vitro using the Prolactin ELISA (Immuno-Biological Laboratories America), and oxytocin production may be assessed in vitro using the Oxytocin OT ELISA Kit (MyBiosource, San Diego, Calif.). In each of the foregoing assays, in certain embodiments, culture medium in which the FPUs are cultured is assayed for production of the particular hormone by said FPUs.

In specific embodiments, said pituitary FPUs comprise one or more of pituitary somatotrophs, pituitary mammotrophs, pituitary corticotrophs, pituitary thyrotrophs, pituitary gonadotrophs, and/or pituitary neurosecretory cells. In certain other specific embodiments, the pituitary FPUs can comprise (e.g., can also comprise), cells that have been genetically engineered to produce one or more pituitary-specific hormones. In certain specific embodiments, the FPUs further comprise vascular endothelial cells, wherein said vascular endothelial cells are arranged within said FPUs to define one or more vessels. In more specific embodiments, said one or more vessels are capable of containing blood or lymph. In other more specific embodiments, said FPUs are constructed so that said one or more of pituitary somatotrophs, pituitary mammotrophs, pituitary corticotrophs, pituitary thyrotrophs, pituitary gonadotrophs, and/or pituitary neurosecretory cells are positioned adjacent to one or more of said vessels. In certain specific embodiments, the at least one vessels are constructed to allow entrance of blood into said FPUs, and exit of blood from said FPUs, e.g., entrance by a single entrance vessel and/or exit by a single exit vessel. In certain specific embodiments, said vessels are constructed to form an anastomosing network of vessels, in which two of more of said vessels split from said entrance vessel and rejoin at a point prior to said exit vessel.

In certain other embodiments, said one or more of pituitary somatotrophs, pituitary mammotrophs, pituitary corticotrophs, pituitary thyrotrophs, pituitary gonadotrophs, and/or pituitary neurosecretory cells are positioned at or adjacent to the exterior surface of said FPUs, such that cells can take up nutrients from the exterior of the FPUs by diffusion, and said one or more pituitary-specific hormones can diffuse from said FPUs into the surrounding environment, e.g., into culture medium or into an individual into which said FPUs are implanted.

### 4.4.2 Thyroid Gland

The thyroid comprises thyroid follicular cells, which secrete colloid; thyroid epithelial cells, which produce T3 and T4; and thyroid parafollicular cells, which produce calcitonin. In certain embodiments, therefore, provided herein are FPUs that perform at least one physiological function of a thyroid gland, e.g., provided herein are thyroid FPUs. In specific embodiments, said at least one physiological function of a thyroid is, or said thyroid FPUs produce, detectable amounts of one or more thyroid-specific hormones, e.g., one or more of triiodothyronine (T3), thyroxine (T4) and/or calcitonin. Production of said one or more thyroid-specific hormones by said FPUs may be assayed, e.g., by commercially-available kits and assays. For example, T3 production may be assayed in vitro using the Total T3 ELISA Kit (MyBiosource, San Diego, Calif.); T4 production may be assayed in vitro using the Total T4 ELISA Kit (MyBiosource, San Diego, Calif.); and calcitonin production may be assayed in vitro using the Calcitonin ELISA Kit (MyBiosource, San Diego, Calif.). In certain embodiments, said FPUs comprise (e.g., additionally comprises), cells that have been genetically engineered to produce detectable amounts of one or more thyroid-specific hormones, e.g., one or more of T3, T4 and/or calcitonin. In each of the foregoing assays, in certain embodiments, culture medium in which the FPUs are cultured is assayed for production of the particular hormone by said FPUs.

In specific embodiments, said thyroid FPUs comprise one or more of thyroid follicular cells, thyroid epithelial cells, and/or thyroid parafollicular cells. In another specific embodiment, said thyroid FPUs comprise thyroid follicular cells arranged as a circle or ball of cells around a central portion lacking cells so as to form an artificial follicle. In a more specific embodiment, said FPUs comprise a plurality of artificial follicles. In a more specific embodiment, said FPUs comprise a layer of thyroid epithelial cells partially or completely surrounding said artificial follicle. In a more specific embodiment, said FPUs comprise thyroid parafollicular cells in addition to said artificial follicle and said thyroid epithelial cells.

In certain specific embodiments, the thyroid FPUs further comprise vascular endothelial cells, wherein said vascular endothelial cells are arranged within said FPUs to define one or more vessels. In more specific embodiments, said one or more vessels are capable of containing blood or lymph. In other more specific embodiments, said FPUs are constructed so that at least some, or all, of said artificial follicles are positioned adjacent to one or more of said vessels. In certain specific embodiments, the at least one vessels are constructed to allow entrance of blood into said FPUs, and exit of blood from said FPUs, e.g., entrance by a single entrance vessel and/or exit by a single exit vessel. In certain specific embodiments, said vessels are constructed to form an anastomosing network of vessels, in which two of more of said vessels split from said entrance vessel and rejoin at a point prior to said exit vessel.

In certain other embodiments, said thyroid FPUs are constructed so that one or more of said artificial follicles, thyroid epithelial cells, and/or thyroid parafollicular cells are positioned at or adjacent to the exterior surface of said FPUs, such that cells can take up nutrients from the exterior of the FPUs by diffusion, and said one or more thyroid-specific hormones can diffuse from said FPUs into the surrounding environment, e.g., into culture medium or into an individual into which said FPUs is implanted.

### 4.4.3 Parathyroid Gland

The parathyroid gland primarily comprises two types of cells: parathyroid chief cells, responsible for the production of parathyroid hormone, and parathyroid oxyphil cells. In certain embodiments, therefore, provided herein are FPUs that perform at least one physiological function of a parathyroid gland, e.g., provided herein are parathyroid FPUs. In specific embodiments, said at least one physiological function of a parathyroid gland is production of, or said parathyroid FPUs produce, detectable amounts of parathyroid hormone (PTH). Production of PTH can be assessed in vitro, e.g. by testing culture medium in which said FPUs are cultured, for the presence of PTH using the Intact-PTH ELISA Kit (Immuno-Biological Laboratories, Minneapolis, Minn.). In certain embodiments, the parathyroid FPUs comprise parathyroid chief cells. In more specific embodiments, the parathyroid FPUs comprise both parathyroid chief cells and parathyroid oxyphil cells. In certain embodiments, said FPUs comprise (e.g., additionally comprises), cells that have been genetically engineered to produce detectable amounts of PTH. In each of the foregoing assays, in certain embodiments, culture medium in which the FPUs are cultured is assayed for production of the particular hormone or protein by said FPUs.

In certain specific embodiments, the parathyroid FPUs further comprise vascular endothelial cells, wherein said vascular endothelial cells are arranged within said FPUs to define one or more vessels. In more specific embodiments, said at least one vessel is capable of containing blood or lymph. In other more specific embodiments, said FPUs are constructed so that said parathyroid chief cells and/or said parathyroid oxyphil cells arc positioned adjacent to one or more of said vessels. In certain specific embodiments, the at least one vessels are constructed to allow entrance of blood into said FPUs, and exit of blood from said FPUs, e.g., entrance by a single entrance vessel and/or exit by a single exit vessel. In certain specific embodiments, said vessels are constructed to form an anastomosing network of vessels, in which two of more of said vessels split from said entrance vessel and rejoin at a point prior to said exit vessel.

In certain other embodiments, parathyroid chief cells and/or said parathyroid oxyphil cells are positioned at or adjacent to the exterior surface of said FPUs, such that cells can take up nutrients from the exterior of the FPUs by diffusion, and said one or more pituitary-specific hormones can diffuse from said FPUs into the surrounding environment, e.g., into culture medium or into an individual into which said FPUs is implanted.

### 4.4.4 Adrenal Gland

The adrenal gland comprises adrenal chromaffin cells, which are primarily responsible for production of epinephrine; adrenal zona glomerulosa cells, which produce mineralocorticoids (primarily aldosterone); adrenal zona fasciculata cells, which produce glucocorticoids (e.g., 11-dcoxycorticosterone, corticosterone, and/or cortisol); and adrenal zona reticularis cells, which produce androgens (e.g., dehydroepiandrosterone (DHEA) and/or androstenedione). In certain embodiments, therefore, provided herein are FPUs that perform at least one physiological function of an adrenal gland, e.g., provided herein are adrenal FPUs. In specific embodiments, said at least one physiological function of an adrenal gland is production of, or said adrenal FPUs produce, detectable amounts of one or more adrenal-specific hormones, e.g., one or more of aldosterone, fludrocortisone, dehydroepiandrosterone, 18 hydroxy 11 deoxycorticosterone, corticosterone, cortisol, DHEA and/or androstenedione. In certain embodiments, said FPUs comprise (e.g., additionally comprise), cells that have been genetically engineered to produce detectable amounts of one or more of, e.g., aldosterone, 11-deoxycorticosterone, corticosterone, cortisol, fludrocortisone, DHEA and/or androstenedione.

Production of said one or more adrenal gland-specific hormones may be assayed, e.g., by published and/or commercially-available kits and assays. For example, production of fludrocortisone by said adrenal FPUs can be assessed using a liquid chromatography assay; see Ast et al., J. Pharm. Sci. 68(4):421-423 (1979). Production of aldostcronc by the adrenal FPUs can be assayed using the Human Aldosterone ELISA Kit (BioVendor Laboratory Medicine, Inc., Candler, N.C.). Production of cortisol by the adrenal FPUs can be assayed by the Cortisol ELISA Kit (Enzo Life Sciences, Inc., Farmingdale, N.Y.). Production of 18 hydroxy 11 deoxycorticosterone by said adrenal FPUs can be assayed using a radioimmune assay; see Chandler et al., Steroids 27; (2):235-246 (1976). Production of epinephrine by said adrenal FPUs may be assayed by the Epinephrine RIA (Alpco Diagnostics, Salem, N.H.). Androstenedione production by said adrenal FPUs can be assayed by mass spectrometry; see Booker et al., Drug Testing and Analysis 1(11-12):587-595 (2009). DHEA production by the adrenal FPUs may be assayed by the DHEA ELISA kit (Abnova Corporation, Taipei City, Taiwan). In each of the foregoing assays, in certain embodiments, culture medium in which the FPUs are cultured is assayed for production of the particular hormone or protein by said FPUs.

In certain specific embodiments, the adrenal FPUs comprise adrenal chromaffin cells, adrenal zona fasciculata cells, adrenal zona glomerulosa cells, and/or adrenal zona reticularis cells. In a specific embodiment, said adrenal FPUs comprise two or more of adrenal zona fasciculata cells, adrenal zona glomerulosa cells, and/or adrenal zona reticularis cells. In certain specific embodiments, said adrenal chromaffin cells, adrenal zona fasciculata cells, adrenal zona glomerulosa cells, and/or adrenal zona reticularis cells are arranged randomly, or are regularly ordered, within said adrenal FPUs. In certain other specific embodiments, said adrenal chromaffin cells are grouped together within said FPUs, said adrenal zona fasciculata cells are grouped together within said FPUs, said adrenal zona glomerulosa cells are grouped together within said FPUs, and/or adrenal zona reticularis cells are grouped together within said adrenal FPUs. In another specific embodiment, said adrenal FPUs comprises zona glomerulosa cells and zona fasciculata cells, wherein said zona glomerulosa cells and zona fasciculata cells are separate from each other in said adrenal FPUs. In another specific embodiment, said adrenal FPUs comprise zona glomerulosa cells and zona reticularis cells, wherein said zona glomerulosa cells and zona reticularis cells are separate from each other in said adrenal FPUs. In another said adrenal FPUs comprise zona reticularis cells and zona fasciculata cells, wherein said zona reticularis cells and zona fasciculata cells are separate from each other in said adrenal FPUs. In another specific embodiment, the adrenal FPUs comprise zona glomerulosa cells, zona fasciculata cells, and zona reticularis cells, wherein each of said zona glomerulosa cells, zona fasciculata cells, and zona reticularis cells are each separate from the other cell types in said adrenal FPUs.

In certain specific embodiments, the adrenal FPUs further comprise vascular endothelial cells, wherein said vascular endothelial cells are arranged within said FPUs to define one or more vessels. In more specific embodiments, said one or more vessels are capable of containing blood or lymph. In other more specific embodiments, said FPUs are constructed so that at least some, or all, of said artificial follicles are positioned adjacent to one or more of said vessels. In certain specific embodiments, the at least one vessels are constructed to allow entrance of blood into said FPUs, and exit of blood from said FPUs, e.g., entrance by a single entrance vessel and/or exit by a single exit vessel. In certain specific embodiments, said vessels are constructed to form an anastomosing network of vessels, in which two of more of said vessels split from said entrance vessel and rejoin at a point prior to said exit vessel.

In certain other embodiments, said adrenal FPUs are constructed so that one or more of said adrenal chromaffin cells, adrenal zona glomerulosa cells, adrenal zona fasciculata cells, and/or adrenal zona reticularis cells are positioned at or adjacent to the exterior surface of said FPUs, such that cells can take up nutrients from the exterior of the FPUs by diffusion, and said one or more thyroid-specific hormones can diffuse from said FPUs into the surrounding environment, e.g., into culture medium or into an individual into which said FPUs are administered or implanted.

### 4.4.5 Pancreas

The pancreas comprises pancreatic alpha cells, pancreatic beta cells, pancreatic delta cells, pancreatic PP cells, and pancreatic epsilon cells. In certain embodiments, therefore, provided herein are FPUs that perform at least one physiological function of a pancreas, e.g., provided herein are pancreatic FPUs. In specific embodiments, said at least one physiological function of a pancreas is production of, or said pancreatic FPUs produce, detectable amounts of a pancreas-specific hormone or protein, e.g., amylin (also known as islet amyloid polypeptide, or IAPP), insulin, somatostatin, grehlin, pancreatic polypeptide, and/or glucagon, e.g., in vitro. In a more specific embodiment, said FPUs produce insulin and amylin, in vitro, in a ratio of about 10:1, 60:1, 70:1, 80:1, 90:1, 100:1, 110:1, 120:1, 130:1, 140:1, 150:1, 160:1, 170:1, 180:1, 190:1 or 200:1. In certain embodiments, said FPUs comprise (e.g., additionally comprise), cells that have been genetically engineered to produce detectable amounts of one or more of amylin, insulin, glucagon, somatostatin, grchlin, an/or pancreatic polypcptidc.

Production of said one or more pancreas-specific hormones by said pancreatic FPUs can be assayed using commercially-available assays or kits. For example, production of insulin by said pancreatic FPUs in vitro may be assayed by any commonplace insulin test kits; production of glucagon by said pancreatic FPUs in vitro may be assayed by the ELISA Kit for Glucagon (Uscn Life Science, Inc., Wuhan, China); production of somatostatin by the pancreatic FPUs in vitro may be assayed by the Human Somatostatin (SST) ELISA (Kamiya Biomedical Company, Seattle, Wash.); production of grehlin by the pancreatic FPUs in vitro may be assayed by the Grehlin (Human, Mouse, Rat) ELISA Kit (Abnova, Taipei City, Taiwan); production of pancreatic polypeptide by the pancreatic FPUs in vitro may be assayed by the Human Pancreatic Polypeptide (PP) ELISA Kit (EMD Millipore, Billerica, Me.); and production of amylin by said pancreatic FPUs may be assayed by the IAPP (Human) ELISA Kit (Abnova, Taipei City, Taiwan). In each of the foregoing assays, in certain embodiments, culture medium in which the FPUs are cultured is assayed for production of the particular hormone or protein by said FPUs.

In certain specific embodiments, the adrenal FPUs further comprise vascular endothelial cells, wherein said vascular endothelial cells are arranged within said FPUs to define one or more vessels. In more specific embodiments, said one or more vessels are capable of containing blood or lymph. In other more specific embodiments, said FPUs are constructed so that at least some, or all, of said pancreatic alpha cells, pancreatic beta cells, pancreatic delta cells, pancreatic epsilon cells, and/or said pancreatic PP cells are positioned adjacent to said one or more vessels. In certain specific embodiments, the vessels are constructed to allow entrance of blood into said FPUs, and exit of blood from said FPUs, e.g., entrance by a single entrance vessel and/or exit by a single exit vessel. In certain specific embodiments, said vessels are constructed to form an anastomosing network of vessels, in which two of more of said vessels split from said entrance vessel and rejoin at a point prior to said exit vessel.

In certain other embodiments, said pancreatic FPUs arc constructed so that one or more of said pancreatic alpha cells, pancreatic beta cells, pancreatic delta cells, pancreatic epsilon cells, and/or said pancreatic PP cells are positioned at or adjacent to the exterior surface of said FPUs, such that cells can take up nutrients from the exterior of the FPUs by diffusion, and said one or more thyroid-specific hoimones can diffuse from said FPUs into the surrounding environment, e.g., into culture medium or into an individual into which said FPUs are implanted.

### 4.4.6 Liver

The liver comprises primarily parenchymal hepatocytes, which make up 70%-80% of the liver's mass, along with vascular endothelial cells and Kupffer cells. In certain embodiments, therefore, provided herein are FPUs that perform at least one physiological function of a liver, e.g., provided herein are liver FPUs.

In certain specific embodiments, said FPUs produce a measurable amount of one or more of coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin IIA; blood coagulation factor XIV), protein S and/or antithrombin. In various other embodiments of any of the FPUs provided herein, said FPUs produce detectable amounts of glucose from an amino acid, lactate, glycerol or glycogen. In other embodiments, said FPUs produce detectable amounts of insulin-like growth factor (IGF-1) or thrombopoietin. In other embodiments, said FPUs produce bile. In certain embodiments of any of the FPUs provided herein, said FPUs comprise cells that produce one or more of coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin IIA; blood coagulation factor XIV), protein S, antithrombin, IGF-1 or thrombopoietin. In certain embodiments of any of the FPUs provided herein, said FPUs comprise hepatic vessel endothelial cells. In a specific embodiment, said hepatic vessel endothelial cells are disposed within said FPUs so as to define one or more vessels. In a more specific embodiment, said hepatocytes are disposed along and substantially parallel to said vessels. In a more specific embodiment, a plurality of said vessels are disposed in substantially radial fashion so as to define an exterior and an interior of said FPUs, such that each vessel has a distal and a proximal end. In another more specific embodiment, said FPUs comprise at least one vessel that connects each of said distal ends of said vessels.

Production of said one or more liver-specific hormones by said liver FPUs can be assayed using published commercially-available assays or kits. For example, production of fibrinogen by said liver FPUs can be assayed by the Human Fibrinogen ELISA Kit (AbFrontier Co., Ltd., Seoul, KR); production of prothrombin by said liver FPUs may be assayed by the Prothrombin (Human) ELISA kit (Abnova, Taipei City, Taiwan); production of factor five by said liver-specific FPUs may be assayed by the Zymutest Factor V ELISA (Aniara, Mason, OH); production of proconvertin by said liver FPUs can be assayed by the Factor VII (Proconvertin) Activity assay (Gentaur Molecular Products, Whetstone, London, UK); production of coagulation factor XI by said liver FPUs can be assayed by the Total Human Coagulation Factor XI Antigen Assay (Molecular Innovations, Novi, Mich.); production of prothrombinase by said liver FPUs can be assayed by the ELISA Kit for Coagulation Factor X (Uscn Life Science, Wuhan, China); production of coagulation factor XI by said liver FPUs may be assayed by the Factor XI Human ELISA Kit (ab 108834) (Abcam, Cambridge, Mass.); production of protein C by said liver FPUs may be assayed by the Chromogenic Assay Kit for Plasma Protein C (American Diagnostica, Pfungstadt, Germany); production of protein S by said liver FPUs may be assayed by the Human Free Protein S DLISA Kit (American Diagnostica, Pfungstadt, Germany); production of antithrombin by said liver FPUs may be assayed by the ACTICHROME® Antithrombin III Chromogenic Activity Kit (American Diagnostica, Pfungstadt, Germany); production of IGF-1 by said liver FPUs may be assayed by the Human IGF-1 ELISA Kit (AbFrontier, Co., Ltd., Seoul, KR); and production of thrombopoietin by said liver FPUs may be assessed using the Human TPO Thrombopoietin ELISA Kit (Cell Sciences, Canton, Mass.). In each of the foregoing assays, in certain embodiments, culture medium in which the FPUs are cultured is assayed for production of the particular hormone or protein by said FPUs.

### 4.5 Functional Physiological Units: Methods of Making

In another aspect, provided herein is a method of making a functional physiological unit (FPU), comprising combining an isolated extracellular matrix (ECM) and at least one type of cell, such that said FPU performs at least one function of an organ or tissue from an organ, wherein said FPU is less than about 1000 microliters in volume, and wherein said at least one function of an organ or tissue from an organ is production of a protein, cytokine, interleukin, or small molecule characteristic of at least one cell type from said organ or tissue.

The FPUs provided herein may be produced by any biologically-compatible method capable of depositing cells, e.g., onto a surface, in an organized arrangement. In making the FPUs, single cells, or a plurality of cells, may be deposited at a time. Methods of making the FPUs can encompass use of any of the compositions and/or cells described herein.

### 4.5.1 Bioprinting

In certain embodiments, the FPUs provided herein are produced by bioprinting. “Bioprinting” as used herein generally refers to the deposition of living cells onto a surface using standard or modified printing technology, e.g., ink jet printing technology. Basic methods of depositing cells onto surfaces, and of bioprinting cells, including cells in combination with hydrogels, is described in Warren et al. U.S. Pat. No. 6,986,739, Boland et al. U.S. Pat. No. 7,051,654, Yoo et al. US 2009/0208466 and Xu et al. US 2009/0208577, the disclosures of each of which are incorporated by reference herein in its entirety. Additionally, bioprinters suitable for production of the FPUs provided herein are commercially available, e.g., the 3D-Bioplotter™ from Envisiontec GmbH; and the NovoGen MMX Bioprinter™ from Organovo (San Diego, Calif.). Typically, FPUs produced by bioprinting are produced by printing cells and optionally matrix onto a surface, followed by removal of the finalized FPUs from the surface for further processing or use. In certain embodiments, the surface on which the FPUs are constructed is a non-stick surface, such as TEFLON®, THERMOLON® (a silicon oxide compound), polytetrafluoroethylene (PTFE), perflouoroalkoxy, fluorinated ethylene propylene, or the like.

Typically, in bioprinting, individual droplets of cells and/or compositions having small volumes, e.g., from 0.5 to 500 picoliters per droplet, are deposited onto a surface. In various embodiments, the volume of cells, or composition comprising the cells, is about 0.5, 1, 2, 3, 4, 5, 6, 7, 8, 9, 10, 15, 20, 25, 20, 35, 40, 45, 50, 55, 60, 65, 70, 75, 80, 85, 90, 95 or 100 picoliters, or between about 1 to 90 picoliters, about 5 to 85 picoliters, about 10 to 75 picoliters, about 15 to 70 picoliters, about 20 to 65 picoliters, or about 25 to about 60 picoliters.

In certain embodiments, the cells to be bioprinted for production of the FPUs are contained within a flowable physiologically-acceptable composition, e.g., water, buffer solutions (e.g., phosphate buffer solution, citrate buffer solution, etc.), liquid media (e.g., 0.9N saline solution, Krebs's solution, modified Krebs's solution, Eagle's medium, modified Eagle's medium (MEM), Dulbecco's Modified Eagle's Medium (DMEM), Hank's Balanced Salts, etc.), and the like.

In some embodiments, the composition comprising the cells to be printed comprises a polymerizable monomer. In such embodiments, for example, a polymerization catalyst may be added immediately prior to bioprinting, such that once the cells are printed, the monomer polymerizes, forming a gel that traps and/or physically supports the cells. For example, the composition comprising the cells can comprise acrylamide monomers, whereupon TEMED and Ammonium persulfate, or riboflavin, are added to the composition immediately prior to bioprinting. Upon deposition of the cells in the composition onto a surface, the acrylamide polymerizes, sequestering and supporting the cells.

The bioprinter used for construction of the FPUs preferably includes mechanisms and/or software that enables control of the temperature, humidity, shear force, speed of printing, and firing frequency, by modifications of, e.g., the printer driver software and/or the physical makeup of the printer. Printer software and/or hardware preferably is constructed and/or set to maintain a cell temperature of about 37° C. during printing.

The inkjet printing device may include a two-dimensional or three-dimensional printer. In certain embodiments, the bioprinter comprises a DC solenoid inkjet valve, one or more reservoir for containing the one or more types of cells, e.g., cells in the flowable composition, and/or ECM prior to printing, e.g., connected to the inkjet valve. The bioprinter may have 1, 2, 3, 4, 5, 6, 7, 8, 9, 10 or more reservoirs, e.g., one for each cell type or each ECM used to construct the FPUs. The cells may be delivered from the reservoir to the inkjet valve by air pressure, mechanical pressure, or by other means. Typically, the bioprinter, e.g., the print heads in the bioprinter, is/are computer-controlled such that the one or more cell types, and said ECM, are deposited in a predetermined pattern. Said predetermined pattern can be a pattern that recreates or recapitulates the natural arrangement of said one or more types of cells in an organ or tissue from which the cells are derived or obtained, or a pattern that is different from the natural arrangement of said one or more types of cells.

The inkjet printing device may be a thermal bubble inkjet printer, see, e.g., Niklasen et al. U.S. Pat. No. 6,537,567, or a piezoelectric crystal vibration print head, e.g., using frequencies up to 30 kHz and power sources ranging from 12 to 100 Watts. Bioprintcr print head nozzles, in some embodiments, are each independently between 0.05 and 200 micrometers in diameter, or between 0.5 and 100 micrometers in diameter, or between 10 and 70 micrometers in diameter, or between 20 and 60 micrometers in diameter. In further embodiments, the nozzles are each independently about 40 or 50 micrometers in diameter. Multiple nozzles with the same or different diameters may be used. In some embodiments the nozzles have a circular opening; in other embodiments, other suitable shapes may be used, e.g., oval, square, rectangle, etc., without departing from the spirit of the invention.

In certain embodiments, an anatomical image of the FPUs to be bioprinted is constructed using software, e.g., a computer-aided design (CAD) software program. In a specific embodiment, the design of the FPUs using said CAD program is guided by the anatomical structure of the organ, or portion thereof, corresponding to the cells to be included in the FPU. For example, where the primary cells to be included in the FPUs are hepatocytes, the design of the FPUs may be guided by naturally-occurring radial arrangement of hepatocytes around a central vessel in lobules in the liver.

### 4.6 Isolation and Culture of Cells

Cells useful in the production of the FPUs described elsewhere herein may be isolated from the relevant tissue or organs, e.g., from particular glands, using one or more art-known proteases, e.g., collagenase, dispase, trypsin, LIBERASE, or the like. Organ, e.g., gland tissue may be physically dispersed prior to, during, or after treatment of the tissue with a protease, e.g., by dicing, macerating, filtering, or the like. Cells may be cultured using standard, art-known cell culture techniques prior to production of the FPUs, e.g., in order to produce homogeneous or substantially homogeneous cell populations, to select for particular cell types, or the like.

Isolation, culture, and identification of pituitary gland cells may be performed according to procedures known in the art, e.g., using lipocortin 1 (LC1) as a marker according to the procedures disclosed in Christian et al., “Characterization and localization of lipocortin 1-binding sites on rat anterior pituitary cells by fluorescence-activated cell analysis/sorting and electron microscopy,” Endocrinology 138(12):5341-5351 (1997); see also Kim et al., “Isolation, culture and cell-type identification of adult human pituitary cells,” Acta Neuropathol. 68(3):205-208 (1985); Baranowska et al., “Direct effect of cortistatin on OH release from cultured pituitary cells in the rat,” Neuro Endocrinol Lett. 27(1-2):153-156 (2006).

Isolation, culture, and identification of thyroid gland cells may be performed according to procedures known in the art. See, e.g., Pavlov et al., “Isolation of cells for cytological and cytogenetic studies of the thyroid epithelium,” Morfologiia 130(6):81-83 (2006); Fayet et al., “Isolation of a normal human thyroid cell line: hormonal requirement for thyroglobulin regulation,” Thyroid 12(7):539-546(2002).

Isolation, culture, and identification of adrenal gland cells may be performed according to procedures known in the art. See, e.g., Crcutz, “Isolation of chromaffin granules,” Curr Protoc Cell Biol. Chapter 3:Unit 3.39.1-10 (September 2010); Caroccia et al., “Isolation of human adrenocortical aldosterone-producing cells by a novel immunomagnetic beads method,” Endocrinology 151(3):1375-80 (2010); Fawcett et al., “Isolation and properties in culture of human adrenal capillary endothelial cells,” Biochem Biophys Res Commun. 174(2):903-8 (1991); Notter et al., “Rodent and primate adrenal medullary cells in vitro: phenotypic plasticity in response to coculture with C6 glioma cells or NGF,” Exp Brain Res. 76(1):38-46 (1989).

### 5 METHODS OF USING FUNCTIONAL PHYSIOLOGICAL UNITS

The FPUs provided herein can be used in methods of treating an individual having a particular disease or disorder treatable by replacement of, or augmentation of, a physiological function, e.g., production of a biomolecule, e.g., protein or polypeptide, hormone, cytokine, interleukin, interferon, receptors for any of the foregoing, or the like, by administration of FPUs that produce such biomolecule, e.g., and which, when administered, replaces or augments the naturally-occurring biomolecule in the individual. Any of the FPUs provided elsewhere herein can be used for therapeutic purposes, as judged by one of ordinary skill in the art to be appropriate.

Pituitary FPUs, as described above, wherein the FPUs produce one or more pituitary hormones in an individual to whom they are administered, may be therapeutic where the individual is experiencing a disorder due to lack, or reduced production, of a pituitary hormone. Such disorders may, in various embodiments, relate to abnormally reduced growth, disorders of blood pressure, breast milk production, sex organ function, thyroid gland function, water regulation, and/or temperature regulation.

In one embodiment, provided herein is method of treating an individual in need of human growth hormone (hGH) comprising administering to said individual a therapeutically effective amount of hGH-producing Functional Physiological Units (FPU), e.g., that together produce detectable amounts of hGH in said individual, e.g., the FPUs described in section 4.4.1, above. Production of hGH in said individual can be assessed, e.g., using the Human GH ELISA kit (AbFrontier Co., Ltd.; Seoul, KR) with a sample of the individual's serum post-administration.

In another embodiment, provided herein is method of treating an individual in need of prolactin (PRL) comprising administering to said individual a therapeutically effective amount of PRL-producing FPUs, e.g., that together produce detectable amounts of PRL in said individual, e.g., the FPUs described in Section 4.4.1, above. Production of PRL in said individual can be assessed, e.g., using the Prolactin ELISA (Immuno-Biological Laboratories America) with a sample of the individual's serum post-administration. In specific embodiments, said individual has one or more of metabolic syndrome, arteriogenic erectile dysfunction, premature ejaculation, oligozoospermia, asthenospermia, hypofunction of seminal vesicles, or hypoandrogenism.

In another embodiment, provided herein is a method of treating an individual in need of adrenocorticotropic hormone (ACTH) comprising administering to said individual a therapeutically-effective amount of ACTH-producing FPUs, e.g., that together produce detectable amounts of ACTH in said individual, e.g., the FPUs described in Section 4.4.1, above. Production of ACTH in said individual can be assessed, e.g., using the ACTH (1-39) EIA Kit (Bachem, Torrance, Calif.) with a sample of the individual's serum post-administration. In a specific embodiment, said individual has Addison's disease.

In another embodiment, provided herein is a method of treating an individual in need of melanocyte-stimulating hormone (MSH) comprising administering to said individual a therapeutically effective amount of MSH-producing FPUs, e.g., that together produce detectable amounts of MSH in said individual, e.g., the FPUs described in section 4.4.1, above. Production of MSH in said individual can be assessed, e.g., using the Human/Mouse/Rat MSH EIA Kit (Raybiotech, Inc.; Norcross Ga.) with a sample of the individual's serum post-administration. In a specific embodiment, said individual has Alzheimer's disease.

In another embodiment, provided herein is a method of treating an individual in need of thyroid-stimulating hormone (TSH) comprising administering to said individual a therapeutically-effective amount of TSH-producing FPUs, e.g., that together produce detectable TSH in said individual, e.g., the FPUs described in Section 4.4.1, above. Production of TSH in said individual can be assessed, e.g., using the Human TSH ELISA Kit (Calbiotech, Inc., Spring Valley, Calif.) with a sample of the individual's scrum post-administration. In a specific embodiment, said individual has or manifests cretinism.

In another embodiment, provided herein is a method of treating an individual in need of follicle-stimulating hormone (FSH) comprising administering to said individual a therapeutically-effective amount of FSH-producing FPUs, e.g., that together produce detectable FSH in said individual, e.g., the FPUs described in Section 4.4.1, above. Production of FSH in said individual can be assessed, e.g., using the Human FSH ELISA Kit (Anogen, Mississauga, Ontario, Canada) with a sample of the individual's serum post-administration. In a specific embodiment, said individual has or manifests infertility or azoospermia.

In another embodiment, provided herein is a method of treating an individual in need of leutenizing hormone (LH) comprising administering to said individual a therapeutically-effective amount of LH-producing FPUs, e.g., that together produce detectable LH in said individual, e.g., the FPUs described in Section 4.4.1, above. Production of LH in said individual can be assessed, e.g., using the ELISA Kit for Leutenizing Hormone (Uscn Life Science, Wuhan, China) with a sample of the individual's serum post-administration. In a specific embodiment, said individual has or manifests low testosterone, low sperm count or infertility.

In another embodiment, provided herein is a method of treating an individual in need of antidiuretic hormone (ADH) comprising administering to said individual a therapeutically-effective amount of ADH-producing FPUs, e.g., that together produce detectable ADH in said individual, e.g., the FPUs described in Section 4.4.1, above. Production of ADH in said individual can be assessed using the CLIA Kit for Antidiuretic Hormone (ADH) (Uscn Life Science, Wuhan, China) with a sample of the individual's serum post-administration. In a specific embodiment, said individual has hypothalamic diabetes insipidus.

In another embodiment, provided herein is a method of treating an individual in need of oxytocin comprising administering to said individual a therapeutically-effective amount of oxytocin-producing FPUs, e.g., that together produce detectable oxytocin in said individual, e.g., the FPUs described in Section 4.4.1, above. Production of oxytocin in said individual can be assessed, e.g., using the Oxytocin OT ELISA Kit (MyBiosource, San Diego, Calif.) with a sample of the individual's serum post-administration.

Thyroid FPUs, as described above, wherein the FPUs produce one or more thyroid hormones in an individual to whom they are administered, may be therapeutic where the individual is experiencing a disorder due to lack, or reduced production, of a thyroid hormone. Such disorders may, in various embodiments, relate to reduced metabolism, hypothyroidism, Graves disease, Hashimoto's thyroiditis, and the like.

In another embodiment, provided herein is a method of treating an individual in need of thyroxine (T4) comprising administering to said individual a therapeutically-effective amount of T4-producing FPUs, e.g., that together produce detectable T4 in said individual, e.g., the FPUs described in Section 4.4.2, above. T4 production in said individual may be assessed, e.g., using the Total T4 ELISA Kit (MyBiosource, San Diego, Calif.) with a sample of the individual's serum post-administration. In specific embodiments, said individual has or manifests mental retardation, dwarfism, weakness, lethargy, cold intolerance, or moon face associated with T4 deficiency.

In another embodiment, provided herein is a method of treating an individual in need of triiodothyronine (T3) comprising administering to said individual a therapeutically-effective amount of T3-producing FPUs, e.g., that together produce detectable T3 in said individual, e.g., the FPUs described in Sectionb 4.4.2, above. Production of T3 in said individual can be assessed, e.g., using the Total T3 ELISA Kit (MyBiosource, San Diego, Calif.) with a sample of the individual's serum post-administration. In a specific embodiment, said individual has heart disease. In a more specific embodiment, said individual has a serum concentration of T3 that is less than 3.1 μmol/L.

In another embodiment, provided herein is a method of treating an individual in need of calcitonin comprising administering to said individual a therapeutically-effective amount of calcitonin-producing FPUs, e.g., that together produce detectable calcitonin in said individual, e.g., the FPUs described in Section 4.4.2, above. Production of calcitonin in said individual may be assessed, e.g., using the Calcitonin ELISA Kit (MyBiosource, San Diego, Calif.) with a sample of the individual's serum post-administration. In specific embodiments, said individual has osteoporosis or chronic autoimmune hypothyroidism.

In another embodiment, provided herein is a method of treating an individual in need of parathyroid hormone (PTH) comprising administering to said individual a therapeutically-effective amount of PTH-producing FPUs, e.g., that together produce detectable PTH in said individual, e.g., the FPUs described in Section 4.4.3, above. Production of PTH in said individual may be assessed, e.g., using the Intact-PTH ELISA Kit (Immuno-Biological Laboratories, Minneapolis, Minn.) with a sample of the individual's serum post-administration.

Adrenal FPUs, as described above, wherein the FPUs produce one or more adrenal gland hormones in an individual to whom they are administered, may be therapeutic where the individual is experiencing a disorder due to lack, or reduced production, of an adrenal hormone. Such disorders may, in various embodiments, relate to metabolic activity, fat or carbohydrate utilization, inflammation, Cushing syndrome, and/or dysregulation of salt and water balance.

In another embodiment, provided herein is a method of treating an individual in need of aldosterone comprising administering to said individual a therapeutically-effective amount of aldosterone-producing FPUs, e.g., that together produce detectable aldosterone in said individual, e.g., the FPUs described in Section 4.4.4, above. Production of aldosterone in said individual may be assessed, e.g., using the Human Aldosterone ELISA Kit (BioVendor Laboratory Medicine, Inc., Candler, N.C.) with a sample of the individual's serum post-administration. In specific embodiments, said individual has idiopathic hypoaldosteronism, hypereninemic hypoaldosteronism, or hyporeninemic hypoaldosteronism. In another embodiment, said individual has chronic renal insufficiency.

In another embodiment, provided herein is a method of treating an individual in need of 18 hydroxy 11 deoxycorticosterone comprising administering to said individual a therapeutically-effective amount of 18 hydroxy 11 deoxycorticosterone-producing FPUs, e.g., that together produce detectable 18 hydroxy 11 deoxycorticosterone in said individual, e.g., the FPUs described in Section 4.4.4, above. Production of 18 hydroxy 11 deoxycorticosterone in said individual may be assessed, e.g., using a radioimmune assay, see Chandler et al., Steroids 27(2):235-246 (1976) with a sample of the individual's serum post-administration.

In another embodiment, provided herein is a method of treating an individual in need of fludrocortisone comprising administering to said individual a therapeutically-effective amount of fludrocortisone-producing FPUs, e.g., that together produce detectable fludrocortisone in said individual, e.g., the FPUs described in Section 4.4.4, above. Production of fludrocortisone in said individual may be assessed, e.g., using a liquid chromatography assay, see Ast et al., J. Pharm. Sci. 68(4):421-423 (1979), with a sample of the individual's serum post-administration.

In another embodiment, provided herein is a method of treating an individual in need of cortisol comprising administering to said individual a therapeutically-effective amount of cortisol-producing FPUs, e.g., that together produce detectable cortisol in said individual, e.g., the FPUs described in Section 4.4.4, above. Production of cortisol in said individual may be assessed, e.g., using the Cortisol ELISA Kit (Enzo Life Sciences, Inc., Farmingdale, N.Y.) with a sample if the individual's serum. In specific embodiments, said individual has acute adrenal deficiency, Addison's disease, or hypoglycemia.

In another embodiment, provided herein is a method of treating an individual in need of epinephrine comprising administering to said individual a therapeutically-effective amount of epinephrine-producing FPUs, e.g., that together produce detectable epinephrine in said individual, e.g., the FPUs described in Section 4.4.4, above. Production of epinephrine in said individual can be assessed, e.g., using the Epinephrine RIA (Alpco Diagnostics, Salem, N.H.) with a sample of the individual's serum post-administration.

In another embodiment, provided herein is a method of treating an individual in need of androstenedione comprising administering to said individual a therapeutically-effective amount of androstenedione-producing FPUs, e.g., that together produce detectable androstenedione in said individual, e.g., the FPUs described in Section 4.4.4, above. Production of androstenedione in the individual can be assessed, e.g., using mass spectrometry, see Booker et al., Drug Testing and Analysis 1(11-12):587-595 (2009), with a sample of the individual's serum post-administration.

In another embodiment, provided herein is a method of treating an individual in need of dehydroepiandrosterone (DHEA) comprising administering to said individual a therapeutically-effective amount of DHEA-producing FPUs, e.g., that together produce detectable DHEA in said individual, e.g., the FPUs described in Section 4.4.4, above. Production of DHEA in said individual may be assessed, e.g., using the DHEA ELISA kit (Abnova Corporation, Taipei City, Taiwan) with a sample of the individual's serum post-administration.

Further provided herein is a method of treating an individual in need of a compound, comprising administering a therapeutically-effective amount of compound-producing FPUs to said individual, e.g., that together produce detectable compound in said individual, e.g., the FPUs described in Section 4.4.6 above, wherein said compound is coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinasc); coagulation factor XI (plasma thromboplastin antecedent); protein C (autoprothrombin HA; blood coagulation factor XIV), protein S and/or antithrombin. The presence of these compounds in said individual may be assessed using art-known assays with a sample of the individual's serum post-administration

In another embodiment, provided herein is a method of treating an individual in need of IGF-1, comprising administering to said individual a therapeutically-effective amount of IGF-1-producing FPUs, e.g., that together produce detectable IGF-1 in said individual, e.g., the FPUs described in Section 4.4.6, above. Production of IGF-1 in said individual may be assessed, e.g., using the Human IGF-1 ELISA Kit (AbFrontier, Co., Ltd., Seoul, KR) with a sample of serum from said individual.

In another embodiment, provided herein is a method of treating an individual in need of thrombopoietin (Tpo), comprising administering to said individual a therapeutically-effective amount of Tpo-producing FPUs, e.g., that together produce detectable Tpo in said individual, e.g., the FPUs described in Section 4.4.6, above. Production of Tpo in said individual may be assessed, e.g., using the Human TPO/Thrombopoietin ELISA Kit (Cell Sciences, Canton, Mass.) with a sample of serum from said individual.

In another embodiment, provided herein is a method of treating an individual in need of glucagon, comprising administering to said individual a therapeutically-effective amount of glucagon-producing FPUs, e.g., that together produce detectable glucagon in said individual, e.g., the FPUs described in Section 4.4.5, above. Production of glucagon in said individual may be assessed using art-known assays with a sample of serum from said individual.

In another embodiment, provided herein is a method of treating an individual in need of insulin, comprising administering to said individual a therapeutically-effective amount of insulin-producing FPUs, e.g., that together produce detectable insulin in said individual, e.g., the FPUs described in Section 4.4.5, above. Production of insulin in said individual may be assessed using art-known blood sugar tests with a sample of blood from said individual. In a specific embodiment, said individual has diabetes mellitus.

In another embodiment, provided herein is a method of treating an individual in need of amylin, comprising administering to said individual a therapeutically-effective amount of amylin-producing FPUs, e.g., that together produce detectable amylin in said individual, e.g., the FPUs described in Section 4.4.5, above. Production of amylin in said individual may be assessed, e.g., using the IAPP (Human) ELISA Kit (Abnova, Taipei City, Taiwan) with a sample of scrum from said individual.

In another embodiment, provided herein is a method of treating an individual in need of grehlin, comprising administering to said individual a therapeutically-effective amount of grehlin-producing FPUs, e.g., that together produce detectable grehlin in said individual, e.g., the FPUs described in Section 4.4.5, above. Production of grehlin in said individual may be assessed, e.g., using the Grehlin (Human, Mouse, Rat) ELISA Kit (Abnova, Taipei City, Taiwan) with a sample of serum from said individual.

In another embodiment, provided herein is a method of treating an individual in need of pancreatic polypeptide, comprising administering to said individual a therapeutically-effective amount of pancreatic polypeptide-producing FPUs, e.g., that together produce detectable pancreatic polypeptide in said individual, e.g., the FPUs described in Section 4.4.5, above.

Production of pancreatic polypeptide in said individual may be assessed, e.g., using the Human Pancreatic Polypeptide (PP) ELISA Kit (EMD Millipore, Billerica, Me.) with a sample of serum from said individual.

6. EMBODIMENTS

Embodiment 1: A functional physiological unit (FPU), wherein said FPUs comprise in contiguous form an isolated extracellular matrix (ECM) and at least one type of cell, wherein said FPU performs at least one function of an organ or tissue from an organ, where said FPU is less than about 1000 microliters in volume, wherein said at least one function of an organ or tissue from an organ is production of a protein, growth factor, cytokine, interleukin, or small molecule characteristic of at least one cell type from said organ or tissue, and wherein said FPU is in administrable or injectable form.

Embodiment 2: The FPU of embodiment 1, wherein said FPU is less than about 100 microliters in volume.

Embodiment 3: The FPU of embodiment 1, wherein said FPU is less than about 1 microliter in volume.

Embodiment 4: The FPU of embodiment 1, wherein said FPU is less than about 100 picoliters in volume.

Embodiment 5: The FPU of embodiment 1, wherein said FPU is less than about 10 picoliters in volume.

Embodiment 6: The FPU of embodiment 1, wherein said FPU is less than about 10 millimeters along its longest axis.

Embodiment 7: The FPU of embodiment 1, wherein said FPU is less than about 1 millimeter along its longest axis.

Embodiment 8: The FPU of embodiment 1, wherein said FPU is less than about 100 μM along its longest axis.

Embodiment 9: The FPU of embodiment 1, comprising no more than about 10⁵ cells.

Embodiment 10: The FPU of embodiment 1, comprising no more than about 10⁴ cells.

Embodiment 11: The FPU of embodiment 1, comprising no more than about 10³ cells.

Embodiment 12: The FPU of embodiment 1, comprising no more than about 10² cells.

Embodiment 13: The FPU of embodiment 1, comprising at least one channel traversing said FPU, wherein said channel facilitates diffusion of nutrients and/or oxygen to said cells.

Embodiment 14: The FPU of any of embodiments 1-13, additionally comprising a synthetic matrix.

Embodiment 15: The FPU of embodiment 14, wherein said synthetic matrix stabilizes the three-dimensional structure of said FPU.

Embodiment 16: The FPU of embodiment 14 or embodiment 15, wherein said synthetic matrix comprises a polymer or a thermoplastic.

Embodiment 17: The FPU of embodiment 14 or embodiment 15, wherein said synthetic matrix is a polymer or a thermoplastic.

Embodiment 18: The FPU of embodiment 16 or embodiment 17, wherein said thermoplastic is polycaprolactone, polylactic acid, polybutylene terephthal ate, polyethylene terephthalate, polyethylene, polyester, polyvinyl acetate, or polyvinyl chloride.

Embodiment 19: The FPU of embodiment 16 or embodiment 17, wherein said polymer is polyvinylidine chloride, poly(o-carboxyphenoxy)-p-xylene) (poly(o-CPX)), poly(lactide-anhydride) (PLAA), n-isopropyl acrylamide, acrylamide, pent erythritol diacrylate, polymethyl acrylate, carboxymethylcellulose, or poly(lactic-co-glycolic acid) (PLGA).

Embodiment 20: The FPU of embodiment 16 or embodiment 17, wherein said polymer is polyacrylamide.

Embodiment 21: The FPU of any of embodiments 1-20, wherein said extracellular matrix is placental extracellular matrix.

Embodiment 22: The FPU of any of embodiments 1-20, wherein said extracellular matrix is telopeptide placental collagen.

Embodiment 23: The FPU of any of embodiments 1-20, wherein said extracellular matrix is placental extracellular matrix comprising base-treated and/or detergent treated Type I telopeptide placental collagen that has not been chemically modified or contacted with a protease, wherein said ECM comprises less than 5% fibronectin or less than 5% laminin by weight; between 25% and 92% Type I collagen by weight; between 2% and 50% Type III collagen; between 2% and 50% type IV collagen by weight; and/or less than 40% elastin by weight.

Embodiment 24: The FPU of embodiment 13, wherein said telopeptide placental collagen is base-treated, detergent treated Type I telopeptide placental collagen, wherein said collagen has not been chemically modified or contacted with a protease, and wherein said composition comprises less than 1% fibronectin by weight; less than 1% laminin by weight; between 74% and 92% Type I collagen by weight; between 4% and 6% Type III collagen by weight; between 2% and 15% type IV collagen by weight; and/or less than 12% elastin by weight.

Embodiment 25: The FPU of any of embodiments 1-24, having substantially the shape of a rectangular block, a cube, a sphere, a spheroid, a rod, a cylinder, or a torus.

Embodiment 26: The FPU of any of embodiments 1-25 that comprises voids, communicating with the surface of said FPU, large enough to permit entry or exit of cells.

Embodiment 27: The FPU of any of embodiments 1-25 that comprises voids, communicating with the surface of said FPU, not large enough to permit entry or exit of cells.

Embodiment 28: The FPU of any of embodiments 1-27, wherein said ECM is crosslinked or stabilized.

Embodiment 29: The FPU of any of embodiments 1-28, wherein said ECM is combined with a polymer that stabilizes the three-dimensional structure of said FPU.

Embodiment 30: The FPU of any of embodiments 1-29, wherein said cells comprise natural killer (NK) cells.

Embodiment 31: The FPU of embodiment 30, wherein said NK cells comprise CD56⁺ CD16⁻placental intermediate natural killer (PiNK) cells.

Embodiment 32: The FPU of any of embodiments 1-29, wherein said FPUs comprise dendritic cells.

Embodiment 33: The FPU of any of embodiments 1-29, wherein said FPUs comprise thymocytes.

Embodiment 34: The FPU of any of embodiments 1-29, wherein said FPUs comprise thymocytes, lymphoid cells, epithelial reticular cells, and thymic stromal cells.

Embodiment 35: The FPU of any of embodiments 1-29, wherein said FPUs comprise follicular cells.

Embodiment 36: The FPU of embodiment 35, wherein said FPUs comprise cells that express thyroglobulin.

Embodiment 37: The FPU of embodiment 35 or embodiment 36, wherein said FPU additionally comprises thyroid epithelial cells and parafollicular cells.

Embodiment 38: The FPU of any of embodiments 1-29, wherein said FPUs comprise stem cells or progenitor cells.

Embodiment 39: The FPU of any of embodiments 1-29, wherein said stem cells or progenitor cells are embryonic stem cells, embryonic germ cells, induced pluripotent stem cells, mesenchymal stem cells, bone marrow-derived mesenchymal stem cells, bone marrow-derived mesenchymal stromal cells, tissue plastic-adherent placental stem cells (PDACs), umbilical cord stem cells, amniotic fluid stem cells, amnion derived adherent cells (AMDACs), osteogenic placental adherent cells (OPACs), adipose stem cells, limbal stem cells, dental pulp stem cells, myoblasts, endothelial progenitor cells, neuronal stem cells, exfoliated teeth derived stem cells, hair follicle stem cells, dermal stem cells, parthenogenically derived stem cells, reprogrammed stem cells, amnion derived adherent cells, or side population stem cells.

Embodiment 40: The FPU of any of embodiments 1-29, wherein said FPUs comprise hematopoietic stem cells or hematopoietic progenitor cells.

Embodiment 41: The FPU of any of embodiments 1-29, wherein FPUs comprise tissue culture plastic-adherent CD34⁻, CD10 CD105⁺, and CD200⁺ placental stem cells.

Embodiment 42: The FPU of embodiment 41, wherein said placental stem cells are additionally one or more of CD45⁻, CD80⁻, CD86⁻, or CD90⁺.

Embodiment 43: The FPU of embodiment 42, wherein said placental stem cells are additionally CD45⁻, CD80⁻, CD86⁻, and CD90⁺.

Embodiment 44: The FPU of any of embodiments 41-43, wherein said placental stem cells, when said FPU is implanted into a recipient, suppresses an immune response in said recipient.

Embodiment 45: The FPU of embodiment 32, wherein said placental stem cells suppresses an immune response locally within said recipient.

Embodiment 46: The FPU of any of embodiments 1-29, wherein said FPUs comprise differentiated cells.

Embodiment 47: The FPU of embodiment 34, wherein said differentiated cells comprise endothelial cells, epithelial cells, dermal cells, endodermal cells, mesodermal cells, fibroblasts, osteocytes, chondrocytes, natural killer cells, dendritic cells, hepatic cells, pancreatic cells, or stromal cells.

Embodiment 48: The FPU of embodiment 34, wherein said differentiated cells comprise salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells,

somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell,

blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of car), cells of Claudius, cells of Boettcher, choroid plexus cells, pia-arachnoid squamous cells, pigmented ciliary epithelium cells, nonpigmented ciliary epithelium cells, corneal endothelial cells, peg cells,

respiratory tract ciliated cells, oviduct ciliated cell, uterine endometrial ciliated cells, rete testis ciliated cells, ductulus efferens ciliated cells, ciliated ependymal cells,

epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells,

surface epithelial cells of stratified squamous epithelium, basal cell of epithelia, urinary epithelium cells,

auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells

cholinergic neural cells, adrenergic neural cells, peptidergic neural cells,

inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells,

astrocytes, neurons, oligodendrocytes, spindle neurons,

anterior lens epithelial cells, crystallin-containing lens fiber cells,

hepatocytes, adipocytes, white fat cells, brown fat cells, liver lipocytes,

kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells,

ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells,

red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands,

reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, B cells, natural killer cells,

melanocytes, retinal pigmented epithelial cells,

oogonia/oocytes, spermatids, spermatocytes, spermatogonium cells, spermatozoa, ovarian follicle cells, Sertoli cells, thymus epithelial cell, and/or interstitial kidney cells.

Embodiment 49: The FPU of any of embodiments 1-48, wherein cells in said cellular composition are primary culture cells.

Embodiment 50: The FPU of any of embodiments 1-48, wherein cells in said cellular composition are cells that have been cultured in vitro.

Embodiment 51: The FPU of any of embodiments 1-48, wherein said cells have been genetically engineered to produce a protein or polypeptide not naturally produced by the cell, or have been genetically engineered to produce a protein or polypeptide in an amount greater than that naturally produced by the cell, wherein said cellular composition comprises differentiated cells.

Embodiment 52: The FPU of embodiment 51, wherein said protein or polypeptide is a cytokinc or a peptide comprising an active part thereof.

Embodiment 53: The FPU of embodiment 52, wherein said cytokine is adrenomedullin (AM), angiopoietin (Ang), bone morphogenetic protein (BMP), brain-derived neurotrophic factor (BDNF), epidermal growth factor (EGF), erythropoietin (Epo), fibroblast growth factor (FGF), glial cell line-derived neurotrophic factor (GNDF), granulocyte colony stimulating factor (G-CSF), granulocyte-macrophage colony stimulating factor (GM-CSF), growth differentiation factor (GDF-9), hepatocyte growth factor (HGF), hepatoma derived growth factor (HDGF), insulin-like growth factor (IGF), migration-stimulating factor, myostatin (GDF-8), myelomonocytic growth factor (MGF), nerve growth factor (NGF), placental growth factor (P1GF), platelet-derived growth factor (PDGF), thrombopoietin (Tpo), transforming growth factor alpha (TGF-α), TGF-β, tumor necrosis factor alpha (TNF-α), vascular endothelial growth factor (VEGF), or a Wnt protein.

Embodiment 54: The FPU of embodimeet 52 or embodiment 53, wherein a sufficient number of said FPUs to comprise 1×10⁶ cells produces at least 1.0 to 10 μM said cytokine in in vitro culture in growth medium over 24 hours.

Embodiment 55: The FPU of embodiment 51, wherein said protein or polypeptide is a soluble receptor for AM, Ang, BMP, BDNF, EGF, Epo, FGF, GNDF, G-CSF, GM-CSF, GDF-9 , HGF, HDGF, IGF, migration-stimulating factor, GDF-8 , MGF, NGF, PlGF, PDGF, Tpo, TGF-α, TGF-β, TNF-α, VEGF, or a Wnt protein.

Embodiment 56: The FPU of embodiment 55, wherein a sufficient number of said FPUs to comprise 1×10⁶ cells produces at least 1.0 to 10 μM of said soluble receptor in in vitro culture in growth medium over 24 hours.

Embodiment 57: The FPU of embodiment 51, wherein said protein is an interleukin

Embodiment 58: The FPU of embodiment 42, wherein said interleukin is interleukin-1 alpha (IL-1α), IL-1β, IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, both IL-12 alpha and beta subunits, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23 p40 subunit, IL-23 p19 subunit and IL-23 p40 subunit together, IL-24, IL-25, IL-26, IL-27B, IL-27-p28, IL-27B and IL-27-p28 together, IL-28A, IL-28B, IL-29, IL-30, IL-31, IL-32, IL-33, IL-34, IL-35, IL-36α, IL-36β, IL-36γ.

Embodiment 59: The FPU of embodiment 57 or embodiment 58, wherein a sufficient number of said FPUs to comprise 1×10⁶ cells produces at least 1.0 to 10 μM of said interleukin in in vitro culture in growth medium over 24 hours.

Embodiment 60: The FPU of embodiment 51, wherein said protein or polypeptide is a soluble receptor for IL-1α, IL-1β, IL-1F1, IL-1F2, IL-1F3, IL-1F4, IL-1F5, IL-1F6, IL-1F7, IL- 1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23 p40 subunit, IL-24, IL-25, IL-26, IL-27B, IL-27-p28, IL-28A, IL-28B, IL-29, IL-30, IL-31, IL-32, IL-33, IL-34, IL-35, IL-36α, IL-36β, IL-36γ.

Embodiment 61: The FPU of embodiment 60, wherein a sufficient number of said FPUs to comprise 1×10⁶ cells produces at least 1.0 to 10 μM of said soluble receptor in in vitro culture in growth medium over 24 hours.

Embodiment 62: The FPU of embodiment 51, wherein said protein is an interferon (IFN).

Embodiment 63: The FPU of embodiment 62, wherein said interferon is IFN-α, IFN-β, IFN-γ, IFN-λ1, IFN-λ2, IFN-λ3, IFN-K, IFN-ε, IFN-κ, IFN-τ, IFN-δ, IFN-λ, IFN-ω, or IFN-v.

Embodiment 64: The FPU of embodiment 62 or embodiment 63, wherein a sufficient number of said FPUs to comprise 1×10⁶ cells produces at least 1.0 to 10 μM of said interferon in in vitro culture in growth medium over 24 hours.

Embodiment 65: The FPU of embodiment 51, wherein said protein or polypeptide is a soluble receptor for IFN-α, IFN-β, IFN-γ, IFN-λ1, IFN-λ2, IFN-λ3, IFN-K, IFN-ε, IFN-κ, IFN-τ, IFN-δ, IFN-λ, IFN-ω, or IFN-v.

Embodiment 66: The FPU of embodiment 65, wherein a sufficient number of said FPUs to comprise 1×10⁶ cells produces at least 1.0 to 10 μM of said soluble receptor in in vitro culture in growth medium over 24 hours.

Embodiment 67: The FPU of embodiment 51, wherein said protein is insulin or proinsulin.

Embodiment 68: The FPU of embodiment 55, wherein a sufficient number of said FPUs to comprise 1×10⁶ cells produces at least 1.0 to 10 μM of said insulin in in vitro culture in growth medium over 24 hours.

Embodiment 69: The FPU of embodiment 51, wherein said protein is a receptor for insulin.

Embodiment 70: The FPU of embodiment 67 or embodiment 68, wherein said cells have additionally been genetically engineered to produce one or more of prohormone convertase 1, prohormone convertase 2, or carboxypeptidase E.

Embodiment 71: The FPU of embodiment 51, wherein said protein is leptin (LEP).

Embodiment 72: The FPU of embodiment 71, wherein a sufficient number of said FPUs to comprise 1×10⁶ cells produces at least 1.0 to 10 μM of said leptin in in vitro culture in growth medium over 24 hours.

Embodiment 73: The FPU of embodiment 51, wherein said protein is erythropoietin.

Embodimnet 74: The FPU of embodiment 73, wherein a sufficient number of said FPUs to comprise 1×10⁶ cells produces at least 1.0 to 10 μM of said soluble receptor in in vitro culture in growth medium over 24 hours.

Embodiment 75: The FPU of embodiment 51, wherein said protein is thrombopoietin.

Embodiment 76: The FPU of embodiment 75, wherein a sufficient number of said FPUs to comprise 1×10⁶ cells produces at least 1.0 to 10 μM of said soluble receptor in in vitro culture in growth medium over 24 hours.

Embodiment 77: The FPU of embodiment 51, wherein said protein is tyrosine 3-monooxygenase.

Embodiment 78: The FPU of embodiment 77, wherein a sufficient number of said FPUs to comprise 1×10⁶ cells produces at least 1.0 to 10 μM of L-DOPA in in vitro culture in growth medium over 24 hours.

Embodiment 79: The FPU of embodiment 77 or embodiment 78, wherein said cells are further engineered to express aromatic L-amino acid decarboxylase.

Embodiment 80: The FPU of embodiment 79, wherein a sufficient number of said FPUs to comprise 1×10⁶ cells produces at least 1.0 to 10 μM of dopamine in in vitro culture in growth medium over 24 hours.

Embodiment 81: The FPU of embodiment 51, wherein said protein is a hormone or prohormone.

Embodiment 82: The FPU of embodiment 81, wherein said hormone is antimullcrian hormone (AMH), adiponectin (Acrp30), adrenocorticotropic hormone (ACTH), angiotensin (AGT), angiotensinogen (AGT), antidiuretic hormone (ADH), vasopressin, atrial-natriuretic peptide (ANP), calcitonin (CT), cholecystokinin (CCK), corticotrophin-releasing hormone (CRH), erythropoietin (Epo), follicle-stimulating hormone (FSH), testosterone, estrogen, gastrin (GRP), ghrelin, glucagon (GCG), gonadotropin-releasing hormone (GnRH), growth hormone (GH), growth hormone releasing hormone (GHRH), human chorionic gonadotropin (hCG), human placental lactogen (HPL), inhibin, leutinizing hormone (LH), melanocyte stimulating hormone (MSH), orexin, oxytocin (OXT), parathyroid hormone (PTH), prolactin (PRL), relaxin (RLN), secretin (SCT), somatostatin (SRIF), thrombopoietin (Tpo), thyroid-stimulating hormone (Tsh), and/or thyrotropin-releasing hormone (TRH).

Embodiment 83: The FPU of embodiment 51, wherein said protein is cytochrome P450 side chain cleavage enzyme (P450SCC).

Embodiment 84: The FPU of embodiment 51, wherein said protein is a protein missing or malfunctioning in an individual who has a genetic disorder or disease.

Embodiment 85: The FPU of embodiment 84, wherein:
said genetic disease is familial hypercholesterolemia and said protein is low density lipoprotein receptor (LDLR);
said genetic disease is polycystic kidney disease, and said protein is polycystin-1 (PKD1), PKD-2 or PKD3;
said genetic disease is phenylketonuria and said protein is phenylalanine hydroxylase;

Embodiment 86: The FPU of any of embodiments 1-85, wherein said FPUs comprise an immune suppressive compound or an anti-inflammatory compound.

Embodiment 87: The FPU of embodiment 86, wherein said compound is a non-steroidal anti-inflammatory drug (NSAID), acetaminophen, naproxen, ibuprofen, acetylsalicylic acid, a steroid, an anti-T cell receptor antibody, an anti-IL-2 receptor antibody, basiliximab, daclizumab (ZENAPAX®), anti T cell receptor antibodies (e.g., Muromonab-CD3), azathioprine, a corticosteroid, cyclosporine, tacrolimus, mycophenolate mofetil, sirolimus, calcineurin inhibitors, and the like.

Embodiment 88: The FPU of any of embodiments 1-87, wherein said FPU dissolves or degrades within a recipient of the FPU.

Embodiment 89: The FPU of any of embodiments 1-87, wherein said FPU maintains its structural integrity within a recipient of the FPU.

Embodiment 90: The FPU of any of embodiments 1-87, wherein said FPU performs at least one function of a liver, kidney, pancreas, thyroid or lung.

Embodiment 91: The FPU of any of embodiments 1-29, comprising pituitary gland acidophil cells.

Embodiment 92: The FPU of any of embodiments 1-29, comprising pituitary basophil cells.

Embodiment 93: The FPU of any of embodiments 1-19, 91 or 92, comprising both pituitary gland acidophil cells and basophil cells.

Embodiment 94: The FPU of embodiment 91 or embodiment 93, comprising pituitary somatotropes.

Embodiment 95: The FPU of embodiment 91 or embodiment 93, comprising pituitary mammotrophs.

Embodiment 96: The FPU of embodiment 92 or embodiment 93, comprising pituitary corticotrophs.

Embodiment 97: The FPU of embodiment 92 or embodiment 93, comprising pituitary thyrotrophs.

Embodiment 98: The FPU of embodiment 92 or embodiment 93, comprising pituitary gonadotrophs.

Embodiment 99: The FPU of any of embodiments 91-98, wherein said FPUs comprise two or more of pituitary somatotrophs, pituitary mammotrophs, pituitary corticotrophs, pituitary thyrotrophs, and/or pituitary gonadotrophs.

Embodiment 100: The FPU of any of embodiments 91-99, wherein said FPUs produce a measurable amount of growth hormone (GH) in in vitro culture.

Embodiment 101: The FPU of any of embodiments 91-99, wherein said FPUs produce a measurable amount of somatotrophic hormone (STH) in in vitro culture.

Embodiment 102: The FPU of any of embodiments 91-99, wherein said FPUs produce a measurable amount of prolactin (PRL) in in vitro culture.

Embodiment 103: The FPU of any of embodiments 91-99, wherein said FPUs produce a measurable amount of adrenocorticotropic hormone (ACTH) in in vitro culture.

Embodiment 104: The FPU of any of embodiments 91-99, wherein said FPUs produce a measurable amount of melanocyte-stimulating hormone (MSH) in in vitro culture.

Embodiment 105: The FPU of any of embodiments 91-99, wherein said FPUs produce a measurable amount of thyroid-stimulating hormone (TSH) in in vitro culture.

Embodiment 106: The FPU of any of embodiments 91-99, wherein said FPUs produce a measurable amount of follicle-stimulating hormone (FSH) in in vitro culture.

Embodiment 107: The FPU of any of embodiments 91-99, wherein said FPUs produce a measurable amount of leutinizing hormone (LH) in in vitro culture.

Embodiment 108: The FPU of any of embodiments 1-29 or 91-108, wherein said FPUs comprise cells that produce one or more of GH, STH, PRL, ACTH, MSH, TSH, FSH, and/or LH.

Embodiment 109: The FPU of embodiment 108, wherein said cells have been genetically engineered to produce one or more of GH, STH, PRL, ACTH, MSH, TSH, FSH, and/or LH.

Embodiment 110: The FPU of any of embodiments 1-29 comprising hypothalamic neurons.

Embodiment 111: The FPU of nay of embodiments 1-29 comprising pituicytes.

Embodiment 112: THE FPU of embodiment 110 or embodiment 111 comprising both hypothalamic neurons and pituicytes.

Embodiment 113: The FPU of any of embodiments 110-112, wherein said FPUs produce a measurable amount of antidiuretic hormone (ADH) in in vitro culture.

Embodiment 114: The FPU of any of embodiments 110-112, wherein said FPUs produce a measurable amount of oxytocin in in vitro culture.

Embodiment 115: The FPU of any of embodiments 1-29 or 110-112, wherein said FPUs comprise cells that produce one or both of ADH and/or oxytocin.

Embodiment 116: The FPU of embodiment 115, wherein said FPUs comprise cells that have been genetically engineered to produce one or both of ADH and/or oxytocin.

Embodiment 117: The FPU of any of embodiments 91-116 comprising endothelial vessel-forming cells.

Embodiment 118: The FPU of embodiment 117, wherein said FPUs comprise a plurality of vessels.

Embodiment 119: The FPU of embodiment 118, wherein said vessels constitute a reticulated network of said vessels.

Embodiment 120: The FPU of any of embodiments 1-29, wherein said FPUs comprise thyroid epithelial cells.

Embodiment 121: The FPU of any of embodiments 1-29, wherein said FPUs comprise thyroid parafollicular cells.

Embodiment 122: The FPU of any of embodiments 1-29, wherein said FPUs comprise thyroglobulin-producing cells.

Embodiment 123: The FPU of any of embodiments 120-122, wherein said FPUs comprise two or more of thyroid epithelial cells, thyroid parafollicular cells, and thyroglobulin-producing cells.

Embodiment 124: The FPU of embodiment 123, wherein said FPUs comprise blood vessels.

Embodiment 125: The FPU of embodiment 123, wherein said FPUs comprise lymphatic vessels.

Embodiment 126: The FPU of any of embodiments 120-125, wherein said FPUs produce a measurable amount of thyroxine (T4) in in vitro culture.

Embodiment 127: The FPU of any of embodiments 120-125, wherein said FPUs produce a measurable amount of triiodothyronine (T3) in in vitro culture.

Embodiment 128: The FPU of any of embodiments 120-125, wherein said FPUs produce a measurable amount of calcitonin.

Embodiment 129: The FPU of any of embodiments 1-19 or 120-128, wherein said FPUs comprise cells that produce one or more of T3, T4 and/or calcitonin.

Embodiment 130: The FPU of embodiment 129, wherein said FPUs comprise cells genetically engineered to produce one or more of T3, T4 and/or calcitonin.

Embodiment 131: The FPU of any of embodiments 1-29, wherein said FPUs comprise parathyroid chief cells.

Embodiment 132: The FPU of any of embodiments 1-29, wherein said FPUs comprise parathyroid oxyphil cells.

Embodiment 133: The PFU of embodiment 131 or embodiment 132, wherein said FPUs comprise both parathyroid chef cells and parathyroid oxyphil cells.

Embodiment 134: The FPU of embodiment 131 or embodiment 132, wherein said FPUs comprise a plurality of vessels.

Embodiment 135: The FPU of any of embodiments 131-134, wherein said FPUs produce a measurable amount of parathyroid hormone (PTH) in in vitro culture.

Embodiment 136: The FPU of any of embodiments 1-19 or 131-135, wherein said FPUs comprise cells that produce PTH.

Embodiment 137: The FPU of embodiment 136, wherein said FPUs comprise cells that have been genetically engineered to produce said PTH.

Embodiment 138: The FPU of any of embodiments 1-29, wherein said FPUs comprise adrenal gland zona glomerulosa cells.

Embodiment 139: The FPU of any of embodiments 1-29, wherein said FPUs comprise adrenal gland fasciculate cells.

Embodiment 140: The FPU of any of embodiments 1-29, wherein said FPUs comprise adrenal gland zona reticulata cells.

Embodiment 141: The FPU of any of embodiments 1-29, wherein said FPUs comprise adrenal gland chromaffin cells.

Embodiment 142: The FPU of any of embodiments 138-141 comprising vessels.

Embodiment 143: The FPU of any of embodiments 131-142, wherein said FPUs produce a measurable amount of aldosterone in in vitro culture.

Embodiment 144: The FPU of any of embodiments 131-142, wherein said FPUs produce a measurable amount of 18 hydroxy 11 deoxycorticosterone in in vitro culture.

Embodiment 145: The FPU of any of embodiments 131-142, wherein said FPUs produce a measurable amount of fludrocortisone in in vitro culture.

Embodiment 146: The FPU of any of embodiments 131-142, wherein said FPUs produce a measurable amount of cortisol.

Embodiment 147: The FPU of any of embodiments 131-142, wherein said FPUs produce a measurable amount of a non-cortisol glucocorticoid.

Embodiment 148: The FPU of any of embodiments 131-142, wherein said FPUs produce a measurable amount of epinephrine.

Embodiment 149: The FPU of any of embodiments 131-142, wherein said FPUs produce a measurable amount of adrenosterone.

Embodiment 150: The FPU of any of embodiments 131-142, wherein said FPUs produce a measurable amount of dehydroepiandreosterone.

Embodiment 151: The FPU of any of embodiments 1-29 or 131-150, wherein said FPUs comprise cells that produce one or more of aldosterone, 18 hydroxy 11 deoxycorticosterone, cortisol, fludrocortisones, a non-cortisol glucocorticoid, epinephrine, adrenosterone, and/or dehydroepiandrosterone.

Embodiment 152: The FPU of embodiment 151, wherein said FPUs comprise cells that have been genetically engineered to produce one or more of aldosterone, 18 hydroxy 11 deoxycorticosterone, cortisol, fludrocortisones, a non-cortisol glucocorticoid, epinephrine, adrenosterone, and/or dehydroepiandrosterone.

Embodiment 153: The FPU of any of embodiments 1-29, wherein said FPUs comprise hepatocytes.

Embodiment 154: The FPU of embodiment 153, wherein said FPUs produce a measurable amount of one or more of coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin HA; blood coagulation factor XIV), protein S and/or antithrombin.

Embodiment 155: The FPU of embodiment 153, wherein said FPUs produce detectable amounts of glucose from an amino acid, lactate, glycerol or glycogen.

Embodiment 156: The FPU of embodiment 153, wherein said FPUs produce detectable amounts of insulin-like growth factor (IGF-1) or thrombopoietin.

Embodiment 157: The FPU of embodiment 153, wherein said FPUs produce bile.

Embodiment 158: The FPU of any of embodiments 1-29 or 153, wherein said FPUs comprise cells that produce one or more of coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin IIA; blood coagulation factor XIV), protein S, antithrombin, IGF-1 or thrombopoictin.

Embodiment 159: The FPU of any of embodiments 1-29 or 153-158, wherein said FPUs comprise hepatic vessel endothelial cells.

Embodiment 160: The FPU of embodiment 159, wherein said hepatic vessel endothelial cells are disposed within said FPU so as to define one or more vessels.

Embodiment 161: The FPU of embodiment 160, wherein said hepatocytes are disposed along and substantially parallel to said vessels.

Embodiment 162: The FPU of embodiment 160 or 161, wherein a plurality of said vessels are disposed in substantially radial fashion so as to define an exterior and an interior of said FPU, such that each vessel has a distal and a proximal end.

Embodiment 163: The FPU of embodiment 162, wherein said FPUs comprise at least one vessel that connects each of said distal ends of said vessels.

Embodiment 164: The FPU of any of embodiments 1-29, wherein said FPUs comprise pancreatic alpha cells.

Embodiment 165: The FPU of any of embodiments 1-29, wherein said FPUs comprise pancreatic beta cells.

Embodiment 166: The FPU of any of embodiments 1-29, wherein said FPUs comprise pancreatic delta cells.

Embodiment 167: The FPU of any of embodiments 1-29, wherein said FPUs comprise pancreatic PP cells.

Embodiment 168: The FPU of any of embodiments 1-29, wherein said FPUs comprise pancreatic epsilon cells.

Embodiment 169: The FPU of any of embodiments 1-29 or 164-168, wherein said FPUs comprise two or more of pancreatic alpha cells, pancreatic beta cells, pancreatic delta cells, pancreatic PP cells, and/or pancreatic epsilon cells.

Embodiment 170: The FPU of any of any of embodiments 1-19 or 164-169, wherein said FPUs produce a detectable amount of glucagon.

Embodiment 171: The FPU of any of any of embodiments 1-19 or 164-169, wherein said FPUs produce a detectable amount of insulin.

Embodiment 172: The FPU of any of any of embodiments 1-19 or 164-169, wherein said FPUs produce a detectable amount of amylin

Embodiment 173: The FPU of any of any of embodiments 1-19 or 164-169, wherein said FPUs produce a detectable amount of insulin and a detectable amount of amylin.

Embodiment 174: The FPU of embodiment 173, wherein said FPU produces said insulin and said amylin in a ratio of about 50:1 to about 200:1.

Embodiment 175: The FPU of any of any of embodiments 1-19 or 164-169, wherein said FPUs produce a detectable amount of somatostatin.

Embodiment 176: The FPU of any of any of embodiments 1-19 or 164-169, wherein said FPUs produce a detectable amount of grehlin.

Embodiment 177: The FPU of any of any of embodiments 1-19 or 164-169, wherein said FPUs produce a detectable amount of pancreatic polypeptide.

Embodiment 178: The FPU of any of any of embodiments 1-19 or 164-177, wherein said FPUs comprise cells that produce a detectable amount of one or more of insulin, glucagon, amylin, somatostatin, pancreatic polypeptide, and/or grehlin.

Embodiment 179: A method of making a functional physiological unit (FPU), comprising combining an isolated extracellular matrix (ECM) and at least one type of cell, such that said FPU performs at least one function of an organ or tissue from an organ, wherein said FPU is less than about 1000 microliters in volume, and wherein said at least one function of an organ or tissue from an organ is production of a protein, cytokine, interleukin, or small molecule characteristic of at least one cell type from said organ or tissue.

Embodiment 180: The method of embodiment 179, wherein said FPU is less than about 100 microliters in volume.

Embodiment 181: The method of embodiment 179, wherein said FPU is less than about 1 microliter in volume.

Embodiment 182: The method of embodiment 179, wherein said FPU is less than about 100 picoliters in volume.

Embodiment 183: The method of embodiment 179, wherein said FPU is less than about 10 picoliters in volume.

Embodiment 184: The method of embodiment 179, wherein said FPU is less than about 10 millimeters along its longest axis.

Embodiment 185: The method of embodiment 179, wherein said FPU is less than about 1 millimeter along its longest axis.

Embodiment 186: The method of embodiment 179, wherein said FPU is less than about 100 μM along its longest axis.

Embodiment 187: The method of embodiment 179, wherein said FPUs comprise no more than about 10⁵ cells.

Embodiment 188: The method of embodiment 179, wherein said FPUs comprise no more than about 10⁴ cells.

Embodiment 189: The method of embodiment 179, wherein said FPUs comprise no more than about 10³ cells.

Embodiment 190: The method of embodiment 179, wherein said FPUs comprise no more than about 10² cells.

Embodiment 191: The method of embodiment 179, comprising combining said cells and said ECM so as to provide at least one channel that traverses said FPU, wherein said channel facilitates diffusion of nutrients and/or oxygen to said cells.

Embodiment 192: The method of any of embodiments 179-191, additionally comprising combining said cells and said ECM with a synthetic matrix.

Embodiment 193: The method of embodiment 192, wherein said synthetic matrix stabilizes the three-dimensional structure of said FPU.

Embodiment 194: The method of embodiment 192 or embodiment 193, wherein said synthetic matrix comprises a polymer or a thermoplastic.

Embodiment 195: The method of embodiment 192 or embodiment 193, wherein said synthetic matrix is a polymer or a thermoplastic.

Embodiment 196: The method of embodiment 194 or embodiment 195, wherein said thermoplastic is polycaprolactone, polylactic acid, polybutylene terephthalate, polyethylene terephthalate, polyethylene, polyester, polyvinyl acetate, or polyvinyl chloride.

Embodiment 197: The method of embodiment 194 or embodiment 195, wherein said polymer is polyvinylidine chloride, poly(o-carboxyphenoxy)-p-xylene) (poly(o-CPX)), poly(lactide-anhydride) (PLAA), n-isopropyl acrylamide, acrylamide, pent erythritol diacrylate, polymethyl acrylate, carboxymethylcellulose, or poly(lactic-co-glycolic acid) (PLGA).

Embodiment 198: The method of embodiment 194 or embodiment 195, wherein said polymer is polyacrylamide.

Embodiment 199: The method of any of embodiments 179-198, wherein said extracellular matrix is placental extracellular matrix.

Embodiment 200: The method of any of embodiments 179-198, wherein said extracellular matrix is telopeptide placental collagen.

Embodiment 201: The method of any of embodiments 179-198, wherein said extracellular matrix is placental extracellular matrix comprising base-treated and/or detergent treated Type I telopeptide placental collagen that has not been chemically modified or contacted with a protease, wherein said ECM comprises less than 5% fibronectin or less than 5% laminin by weight; between 25% and 92% Type I collagen by weight; between 2% and 50% Type III collagen; between 2% and 50% type IV collagen by weight; and/or less than 40% elastin by weight.

Embodiment 202: The method of embodiment 201, wherein said telopeptide placental collagen is base-treated, detergent treated Type I telopeptide placental collagen, wherein said collagen has not been chemically modified or contacted with a protease, and wherein said composition comprises less than 1% fibronectin by weight; less than 1% laminin by weight; between 74% and 92% Type I collagen by weight; between 4% and 6% Type III collagen by weight; between 2% and 15% type IV collagen by weight; and/or less than 12% elastin by weight.

Embodiment 203: The method of any of embodiments 179-202, wherein said FPU has substantially the shape of a rectangular block, a cube, a sphere, a spheroid, a rod, a cylinder, or a torus.

Embodiment 204: The method of any of embodiments 179-202, wherein said FPU voids, communicating with the surface of said FPU, large enough to permit entry or exit of cells.

Embodiment 205: The method of any of embodiments 179-202, wherein said FPU voids, communicating with the surface of said FPU, not large enough to permit entry or exit of cells.

Embodiment 206: The method of any of embodiments 179-202, wherein said ECM is crosslinked or stabilized.

Embodiment 207: The method of any of embodiments 179-202, wherein said ECM is combined with a polymer that stabilizes the three-dimensional structure of said FPU.

Embodiment 208: The method of any of embodiments 179-207, wherein said combining is performed by printing said cells and aid ECM together.

Embodiment 209: The method of embodiment 208, wherein said printing uses inkjet printing technology.

Embodiment 210: The method of any of embodiments 179-209, wherein at least part of the surface of said FPU is covered with an extracellular matrix or a polymer.

Embodiment 211: The method of any of embodiments 179-209, wherein substantially all of the surface of said FPU is covered with an extracellular matrix or a polymer.

Embodiment 212: The method of any of embodiments 179-209, wherein said combining is performed by adding cells to a hydrophilic solution comprising said ECM; forming a sphere by dropping said solution into a hydrophobic liquid; allowing the ECM in said sphere to harden; and collecting said spheres.

Embodiment 213: The method of any of embodiments 179-212, wherein said at least one type of cells comprises pituitary gland acidophil cells.

Embodiment 214: The method of any of embodiments 179-212, wherein said at least one type of cells comprises pituitary basophil cells.

Embodiment 215: The method of any of embodiments 179-212, wherein said at least one type of cells comprises both pituitary gland acidophil cells and basophil cells.

Embodiment 216: The method of any of embodiments 179-212, wherein said at least one type of cells comprises pituitary somatotrophs.

Embodiment 217: The method of any of embodiments 179-212, wherein said at least one type of cells comprises pituitary mammotrophs.

Embodiment 218: The method of any of embodiments 179-212, wherein said at least one type of cells comprises pituitary corticotrophs.

Embodiment 219: The method of any of embodiments 179-212, wherein said at least one type of cells comprises pituitary thyrotrophs.

Embodiment 220: The method of any of embodiments 179-212, wherein said at least one type of cells comprises pituitary gonadotrophs.

Embodiment 221: The method of any of embodiments 213-220, wherein said FPUs comprise two or more of pituitary somatotrophs, pituitary mammotrophs, pituitary corticotrophs, pituitary thyrotrophs, and/or pituitary gonadotrophs.

Embodiment 222: The method of any of embodiments 213-221, wherein said at least one type of cells comprises vascular endothelial cells.

Embodiment 223: The method of embodiment 222, wherein said vascular endothelial cells are disposed within said FPU so as to form one or more vessels.

Embodiment 224: The method of embodiment 223, where any of said pituitary somatotrophs, pituitary mammotrophs, pituitary corticotrophs, pituitary thyrotrophs, and/or pituitary gonadotrophs are disposed along said vessels during said combining.

Embodiment 225: The method of any of embodiments 213-224, wherein said FPUs produce a measurable amount of growth hormone (GH) in in vitro culture.

Embodiment 226: The method of any of embodiments 213-224, wherein said FPUs produce a measurable amount of somatotrophic hormone (STH) in in vitro culture.

Embodiment 227: The method of any of embodiments 213-224, wherein said FPUs produce a measurable amount of prolactin (PRL) in in vitro culture.

Embodiment 228: The method of any of embodiments 213-224, wherein said FPUs produce a measurable amount of adrenocorticotropic hormone (ACTH) in in vitro culture.

Embodiment 229: The method of any of embodiments 213-224, wherein said FPUs produce a measurable amount of melanocyte-stimulating hormone (MSH) in in vitro culture.

Embodiment 230: The method of any of embodiments 213-224, wherein said FPUs produce a measurable amount of thyroid-stimulating hormone (TSH) in in vitro culture.

Embodiment 231: The method of any of embodiments 213-224, wherein said FPUs produce a measurable amount of follicle-stimulating hormone (FSH) in in vitro culture.

Embodiment 232: The method of any of embodiments 213-224, wherein said FPUs produce a measurable amount of leutinizing hormone (LH) in in vitro culture.

Embodiment 233: The method of any of embodiments 213-224, wherein said FPUs comprise cells that produce one or more of GH, STH, PRL, ACTH, MSH, TSH, FSH, and/or LH.

Embodiment 234: The method of embodiment 233, wherein said FPUs comprise cells have been genetically engineered to produce one or more of GH, STH, PRL, ACTH, MSH, TSH, FSH, and/or LH.

Embodiment 235: The method of any of embodiments 179-212, wherein said at least one type of cells comprises hypothalamic neurons.

Embodiment 236: The method of any of embodiments 179-212, wherein said at least one type of cells comprises pituicytes.

Embodiment 237: The method of any of embodiments 179-212, wherein said at least one type of cells comprises both hypothalamic neurons and pituicytes.

Embodiment 238: The method of any of embodiments 235-237, wherein said FPUs produce a measurable amount of antidiuretic hormone (ADH) in in vitro culture.

Embodiment 239: The method of any of embodiments 235-237, wherein said FPUs produce a measurable amount of oxytocin in in vitro culture.

Embodiment 240: The method of any of embodiments 235-237, wherein said FPUs comprise cells that produce one or both of ADH and/or oxytocin.

Embodiment 241: The method of embodiment 240, wherein said FPUs comprise cells that have been genetically engineered to produce one or both of ADH and/or oxytocin.

Embodiment 242: The method of any of embodiments 213-241, wherein said at least one type of cells additionally comprises endothelial vessel-forming cells.

Embodiment 243: The method of embodiment 242, wherein said endothelial vessel-forming cells are arranged during formation of said FPU so as to produce a plurality of vessels in said FPU.

Embodiment 244: The method of embodiment 243, wherein said endothelial vessel-forming cells are arranged during formation of said FPU so as to produce a reticulated network of said vessels.

Embodiment 245: The method of any of embodiments 179-212, wherein said at least one type of cells comprises thyroid epithelial cells.

Embodiment 246: The method of any of embodiments 179-212, wherein said at least one type of cells comprises thyroid parafollicular cells.

Embodiment 247: The method of any of embodiments 179-212, wherein said at least one type of cells comprises thyroglobulin-producing cells.

Embodiment 248: The method of any of embodiments 245-247, wherein said at least one type of cells comprises two or more of thyroid epithelial cells, thyroid parafollicular cells, and thyroglobulin-producing cells.

Embodiment 249: The method of any of embodiments 245-247, wherein said at least one type of cells further comprises vascular endothelial cells.

Embodiment 250: The method of embodiment 249, wherein said vascular endothelial cells are arranged, during construction of said FPU, so as to form one or more vessels in said FPU.

Embodiment 251: The method of embodiment 250, wherein said vessels are blood vessels.

Embodiment 252: The method of embodiment 250, wherein said vessels are lymphatic vessels.

Embodiment 253: The method of any of embodiments 245-252, wherein said FPUs produce a measurable amount of thyroxine (T4) in in vitro culture.

Embodiment 254: The method of any of embodiments 245-252, wherein said FPUs produce a measurable amount of triiodothyronine (T3) in in vitro culture.

Embodiment 255: The method of any of embodiments 245-252, wherein said FPUs produce a measurable amount of calcitonin.

Embodiment 256: The method of any of embodiments 179-212 or 245-252, wherein said one or more types of cells comprise cells that produce one or more of T3, T4 and/or calcitonin.

Embodiment 257: The method of embodiment 256, wherein said one or more types of cells comprises cells genetically engineered to produce one or more of T3, T4 and/or calcitonin.

Embodiment 258: The method of any of embodiments 179-212, wherein said one or more types of cells comprises parathyroid chief cells.

Embodiment 259: The method of any of embodiments 179-212, wherein said FPUs comprise parathyroid oxyphil cells.

Embodiment 260: The method of embodiment 258 or embodiment 259, wherein said FPUs comprise both parathyroid chef cells and parathyroid oxyphil cells.

Embodiment 261: The method of any of embodiments 258-260, wherein said one or more types of cells comprises vascular endothelial cells.

Embodiment 262: The method of embodiment 261, wherein said vascular endothelial cells are arranged, during construction of said FPU, so as to form one or more vessels in said FPU.

Embodiment 263: The method of embodiment 261 or embodiment 262, wherein said FPUs comprise a plurality of vessels.

Embodiment 264: The method of any of embodiments 258-263, wherein said FPUs produce a measurable amount of parathyroid hormone (PTH) in in vitro culture.

Embodiment 265: The method of any of embodiments 179-212 or 258-263, wherein said FPUs comprise cells that produce PTH.

Embodiment 266: The method of embodiment 265, wherein said one or more types of cells comprises cells that have been genetically engineered to produce said PTH.

Embodiment 267: The method of any of embodiments 179-212, wherein said one or more types of cells comprises adrenal gland zona glomerulosa cells.

Embodiment 268: The method of any of embodiments 179-212, wherein said one or more types of cells comprises adrenal gland fasciculate cells.

Embodiment 269: The method of any of embodiments 179-212, wherein said one or more types of cells comprises adrenal gland zona reticulata cells.

Embodiment 270: The method of any of embodiments 179-212, wherein said one or more types of cells comprises adrenal gland chromaffin cells.

Embodiment 271: The method of any of embodiments 267-270, wherein said one or more types of cells comprises vascular endothelial cells.

Embodiment 272: The method of embodiment 271, wherein said vascular endothelial cells are arranged, during construction of said FPU, so as to form one or more vessels in said FPU.

Embodiment 273: The method of any of embodiments 267-272, wherein said FPUs produce a measurable amount of aldosterone in in vitro culture.

Embodiment 274: The method of any of embodiments 267-272, wherein said FPUs produce a measurable amount of 18 hydroxy 11 deoxycorticosterone in in vitro culture. The method of any of embodiments 267-272, wherein said FPUs produce a measurable amount of fludrocortisone in in vitro culture.

Embodiment 275: The method of any of embodiments 267-272, wherein said FPUs produce a measurable amount of cortisol.

Embodiment 276: The method of any of embodiments 267-272, wherein said FPUs produce a measurable amount of a non-cortisol glucocorticoid.

Embodiment 277: The method of any of embodiments 267-272, wherein said FPUs produce a measurable amount of epinephrine.

Embodiment 278: The method of any of embodiments 267-272, wherein said FPUs produce a measurable amount of adrenosterone.

Embodiment 279: The method of any of embodiments 267-272, wherein said FPUs produce a measurable amount of dehydroepiandreosterone.

Embodiment 280: The method of any of embodiments 179-212 or 267-279, wherein said one or more types of cells comprises cells that produce one or more of aldosterone, 18 hydroxy 11 deoxycorticosterone, cortisol, fludrocortisones, a non-cortisol glucocorticoid, epinephrine, adrenosterone, and/or dehydroepiandrosterone.

Embodiment 281: The FPU of embodiment 281, wherein said one or more types of cells comprises cells that have been genetically engineered to produce one or more of aldosterone, 18 hydroxy 11 deoxycorticosterone, cortisol, fludrocortisones, a non-cortisol glucocorticoid, epinephrine, adrenosterone, and/or dehydroepiandrosterone.

Embodiment 282: The method of any of embodiments 267-281, wherein said one or more types of cells comprises endothelial progenitor cells.

Embodiment 283: The method of embodiment 283, wherein said vascular endothelial cells are arranged, during construction of said FPU, so as to form one or more vessels in said FPU.

Embodiment 284: The method of embodiment 282 or embodiment 283, wherein said FPUs comprise a plurality of vessels.

Embodiment 285: The method of any of embodiments 179-212, wherein said one or more types of cells comprises hepatocytes.

Embodiment 286: The method of embodiment 285 wherein said FPUs produce a measurable amount of one or more of coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin HA; blood coagulation factor XIV), protein S and/or antithrombin.

Embodiment 287: The method of embodiment 285,wherein said FPUs produce detectable amounts of glucose from an amino acid, lactate, glycerol or glycogen.

Embodiment 288: The method of embodiment 285, wherein said FPUs produce detectable amounts of insulin-like growth factor (IGF-1) or thrombopoietin.

Embodiment 289: The method of embodiment 285, wherein said FPUs produce bile.

Embodiment 290: The method of any of embodiments 179-212 or 286-289, wherein said one or more types of cells comprises cells that produce one or more of coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin IIA; blood coagulation factor XIV), protein S, antithrombin, IGF-1 or thrombopoietin.

Embodiment 291: The method of any of embodiments 179-212 or 286-290, wherein said one or more types of cells additionally comprises hepatic vessel endothelial cells.

Embodiment 292: The method of embodiment 291, wherein said hepatic vessel endothelial cells are disposed within said FPU so as to define one or more vessels.

Embodiment 293: The method of embodiment 292, wherein said hepatocytes are disposed along and substantially parallel to said vessels.

Embodiment 294: The method of embodiment 292 or embodiment 293, wherein a plurality of said vessels are disposed in substantially radial fashion so as to define an exterior and an interior of said FPU, such that each vessel has a distal and a proximal end.

Embodiment 295: The method of embodiment 294, wherein said FPUs comprise at least one vessel that connects each of said distal ends of said vessels.

Embodiment 296: The method of any of embodiments 179-212, wherein said one or more types of cells comprises pancreatic alpha cells.

Embodiment 297: The method of any of embodiments 179-212, wherein said one or more types of cells comprises pancreatic beta cells.

Embodiment 298: The method of any of embodiments 179-212, wherein said one or more types of cells delta cells.

Embodiment 299: The method of any of embodiments 179-212, wherein said one or more types of cells PP cells.

Embodiment 300: The method of any of embodiments 179-212, wherein said one or more types of cells epsilon cells.

Embodiment 301: The method of any of embodiments 179-212 or 297-300, wherein said FPUs comprise two or more of pancreatic alpha cells, pancreatic beta cells, pancreatic delta cells, pancreatic PP cells, and/or pancreatic epsilon cells.

Embodiment 302: The method of any of embodiments 179-212 or 296-301, wherein said FPUs produce a detectable amount of glucagon.

Embodiment 303: The method of any of embodiments 179-212 or 296-301, wherein said FPUs produce a detectable amount of insulin.

Embodiment 304: The method of any of embodiments 179-212 or 296-301, wherein said FPUs produce a detectable amount of amylin.

Embodiment 305: The method of any of embodiments 179-212 or 296-301, wherein said FPUs produce a detectable amount of insulin and a detectable amount of amylin.

Embodiment 306: The method of embodiment 305, wherein said PFU produces said insulin and said amylin in a ratio of about 50:1 to about 200:1.

Embodiment 307: The method of any of embodiments 179-212 or 296-301, wherein said FPUs produce a detectable amount of somatostatin.

Embodiment 308: The method of any of embodiments 179-212 or 296-301, wherein said FPUs produce a detectable amount of grehlin.

Embodiment 309: The method of any of embodiments 179-212 or 296-301, wherein said FPUs produce a detectable amount of pancreatic polypeptide.

Embodiment 310: The method of any of embodiments 179-212 or 296-301, wherein said FPUs comprise cells that produce a detectable amount of one or more of insulin, glucagon, amylin, somatostatin, pancreatic polypeptide, and/or grehlin.

Embodiment 311: A method of treating an individual in need of human growth hormone (hGH) comprising administering to said individual a plurality of the functional physiological unit (FPU) of any of embodiments 100, 108 or 109.

Embodiment 312: A method of treating an individual in need of somatotrophic hormone (STH) comprising administering to said individual a plurality of the FPU of any of embodiments 101, 108 or 109.

Embodiment 313: A method of treating an individual in need of prolactin (PRL) comprising administering to said individual a plurality of the FPU of any of embodiments 102, 108 or 109.

Embodiment 314: The method of embodiment 313, wherein said individual has one or more of metabolic syndrome, arteriogenic erectile dysfunction, premature ejaculation, oligozoospermia, asthenospermia, hypofunction of seminal vesicles, or hypoandrogenism.

Embodiment 315: A method of treating an individual in need of adrenocorticotropic hormone (ACTH) comprising administering to said individual a plurality of the FPU of any of embodiments 103, 108 or 109.

Embodiment 316: The method of embodiment 315, wherein said individual has Addison's disease.

Embodiment 317: A method of treating an individual in need of melanocyte-stimulating hormone (hGH) comprising administering to said individual a plurality of the FPU of any of embodiments 104, 108 or 109.

Embodiment 318: The method of embodiment 317, wherein said individual has Alzheimer's' disease.

Embodiment 319: A method of treating an individual in need of thyroid-stimulating hormone (TSH) comprising administering to said individual a plurality of the FPU of any of embodiments 105, 108 or 109.

Embodiment 320: The method of embodiment 319, wherein said individual has or manifests cretinism.

Embodiment 321: A method of treating an individual in need of follicle-stimulating hormone (FSH) comprising administering to said individual a plurality of the FPU of any of embodiments 106, 108 or 109.

Embodiment 322: The method of embodiment 321, wherein said individual has or manifests infertility or azoospermia.

Embodiment 323: A method of treating an individual in need of leutenizing hormone (LH) comprising administering to said individual a plurality of the FPU of any of embodiments 107, 108 or 109.

Embodiment 324: The method of embodiment 323, wherein said individual has or manifests low testosterone, low sperm count or infertility.

Embodiment 325: A method of treating an individual in need of antidiuretic hormone (ADH) comprising administering to said individual a plurality of the FPU of any of embodiments 113, 115, or 116.

Embodiment 326: The method of embodiment 325, wherein said individual has hypothalamic diabetes insipidus.

Embodiment 327: A method of treating an individual in need of oxytocin comprising administering to said individual the FPU of any of embodiments 113, 115, or 116.

Embodiment 328: A method of treating an individual in need of thyroxine (T4) comprising administering to said individual a plurality of the FPU of any of embodiments 126, 129 or 130.

Embodiment 329: The method of embodiment 328, wherein said individual has or manifests mental retardation, dwarfism, weakness, lethargy, cold intolerance, or moon face.

Embodiment 330: A method of treating an individual in need of triiodothyronine (T3) comprising administering to said individual a plurality of the FPU of any of embodiments 127, 129 or 130.

Embodiment 331: The method of embodiment 330, wherein said individual has heart disease.

Embodiment 332: The method of embodiment 330, wherein said individual has a serum concentration of T3 that is less than 3.1 μmol/L.

Embodiment 333: A method of treating an individual in need of calcitonin comprising administering to said individual a plurality of the FPU of any of embodiments 127, 129 or 130.

Embodiment 334: The method of embodiment 333, wherein said individual has osteoporosis or chronic autoimmune hypothyroidism.

Embodiment 335: A method of treating an individual in need of parathyroid hormone (PTH) comprising administering to said individual a plurality of the FPU of any of embodiments 135-137.

Embodiment 336: A method of treating an individual in need of aldosterone comprising administering to said individual a plurality of the FPU of any of embodiments 143, 151 or 152.

Embodiment 337: The method of embodiment 336, wherein said individual has idiopathic hypoaldosteronism, hypereninemic hypoaldosteronism, or hyporeninemic hypoaldosteronism.

Embodiment 338: The method of embodiment 337, wherein said individual has chronic renal insufficiency.

Embodiment 339: A method of treating an individual in need of 18 hydroxy 11 deoxycorticosterone comprising administering to said individual a plurality of the FPU of any of embodiments 144, 151 or 152.

Embodiment 340: A method of treating an individual in need of fludrocortisone comprising administering to said individual a plurality of the FPU of any of embodiments 145, 151 or 152.

Embodiment 341: A method of treating an individual in need of cortisol comprising administering to said individual a plurality of the FPU of any of embodiments 146, 151 or 152.

Embodiment 342: The method of embodiment 341, wherein said individual has acute adrenal deficiency, Addison's disease, or hypoglycemia.

Embodiment 343: A method of treating an individual in need of a non-cortisol glucocorticoid comprising administering to said individual a plurality of the FPU of any of embodiments 147, 151 or 152.

Embodiment 344: A method of treating an individual in need of epinephrine comprising administering to said individual a plurality of the FPU of any of embodiments 148, 151 or 152.

Embodiment 345: A method of treating an individual in need of adrenosterone comprising administering to said individual a plurality of the FPU of any of embodiments 149, 151 or 152.

Embodiment 346: A method of treating an individual in need of dehydroepiandrosterone comprising administering to said individual a plurality of the FPU of any of embodiments 150, 151 or 152.

Embodiment 347: A method of treating an individual in need of a compound, comprising administering the FPU of embodiment 154 or embodiment 158 to said individual, wherein said compound is coagulation factor I (fibrinogen); coagulation factor II (prothrombin); coagulation factor V (factor five); coagulation factor VII (proconvertin); coagulation factor IX (Christmas factor); coagulation factor X (Stuart-Prower factor; prothrombinase); coagulation factor XI (plasma thromboplastin antecedent); protein C (autoprothrombin IIA; blood coagulation factor XIV), protein S and/or antithrombin.

Embodiment 348: A method of treating an individual in need of IGF-1 comprising administering to said individual a plurality of the FPU of embodiment 156.

Embodiment 349: A method of treating an individual in need of thrombopoietin comprising administering to said individual a plurality of the FPU of embodiment 156.

Embodiment 350: A method of treating an individual in need of glucagon comprising administering to said individual a plurality of the FPU of any of embodiments 170 or 178.

Embodiment 351: A method of treating an individual in need of insulin comprising administering to said individual a plurality of the FPU of any of embodiments 171, 173, 174 or 178.

Embodiment 352: The method of embodiment 351, wherein said individual has diabetes mellitus.

Embodiment 353: A method of treating an individual in need of amylin comprising administering to said individual a plurality of the FPU of any of embodiments 172-174 or 178.

Embodiment 354: A method of treating an individual in need of grehlin comprising administering to said individual a plurality of the FPU of embodiment 176 or embodiment 178.

Embodiment 355: A method of treating an individual in need of pancreatic polypeptide comprising administering to said individual a plurality of the FPU of embodiment 177 or embodiment 178.

### Equivalents:

The compositions and methods disclosed herein are not to be limited in scope by the specific embodiments described herein. Indeed, various modifications of the compositions and methods in addition to those described will become apparent to those of skill in the art from the foregoing description. Such modifications are intended to fall within the scope of the appended claims.

Various publications, patents and patent applications are cited herein, the disclosures of which are incorporated by reference in their entireties.

What is claimed is:

1. A functional physiological unit (FPU), wherein said FPUs comprise in contiguous form an isolated extracellular matrix (ECM) and at least one type of cell, wherein said FPU performs at least one function of an organ or tissue from an organ, where said FPU is less than about 1000 microliters in volume, wherein said at least one function of an organ or tissue from an organ is production of a protein, growth factor, cytokine, interleukin, or small molecule characteristic of at least one cell type from said organ or tissue, and wherein said FPU is in administrable or injectable form.

2. The FPU of claim 1, wherein said FPU is less than about 1 microliter in volume.

3. The FPU of claim 1, wherein said FPU is less than about 100 picoliters in volume.

4. The FPU of claim 1, wherein said FPU is less than about 10 picoliters in volume.

5. The FPU of claim 1, comprising no more than about 10⁵ cells.

6. The FPU of claim 1, comprising no more than about 10⁴ cells.

7. The FPU of claim 1, additionally comprising a synthetic matrix.

8. The FPU of claim 1, wherein said ECM is derived from placenta and comprises about 35-55% collagen and about 10-30% elastin.

9. The FPU of claim 1, wherein said at least one type of cell comprises natural killer (NK) cells.

10. The FPU of claim 9, wherein said NK cells comprise CD56³⁰ CD16 placental intermediate natural killer (PiNK) cells.

11. The FPU of claim 1, wherein said FPU comprises stem cells or progenitor cells.

12. The FPU of claim 11, wherein said stem cells or progenitor cells are embryonic stem cells, embryonic germ cells, induced pluripotent stem cells, mesenchymal stem cells, bone marrow-derived mesenchymal stem cells, bone marrow-derived mesenchymal stromal cells, tissue plastic-adherent placental stem cells (PDACs), umbilical cord stem cells, amniotic fluid stem cells, amnion derived adherent cells (AMDACs), osteogenic placental adherent cells (OPACs), adipose stem cells, limbal stem cells, dental pulp stem cells, myoblasts, endothelial progenitor cells, neuronal stem cells, exfoliated teeth derived stem cells, hair follicle stem cells, dermal stem cells, parthenogenically derived stem cells, reprogrammed stem cells, amnion derived adherent cells, or side population stem cells.

13. The FPU of claim 1, wherein said FPU comprises hematopoietic stem cells or hematopoietic progenitor cells.

14. The FPU of claim 1, wherein FPU comprises tissue culture plastic-adherent CD34⁻, CD10⁺, CD105⁺ and CD200⁺ placental stem cells.

15. The FPU of claims 1-14, wherein said FPU comprises differentiated cells.

16. The FPU of claim 15, wherein said differentiated cells comprise endothelial cells, epithelial cells, dermal cells, endodermal cells, mesodermal cells, fibroblasts, osteocytes, chondrocytes, natural killer cells, dendritic cells, hepatic cells, pancreatic cells, or stromal cells.

17. The FPU of claim 15, wherein said differentiated cells comprise salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells,
somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell,
blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells,
respiratory tract ciliated cells, oviduct ciliated cell, uterine endometrial ciliated cells, rete testis ciliated cells, ductulus efferens ciliated cells, ciliated ependymal cells,
epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells,
surface epithelial cells of stratified squamous epithelium, basal cell of epithelia, urinary epithelium cells,
auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells
cholinergic neural cells, adrenergic neural cells, peptidergic neural cells,
inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells,
astrocytes, neurons, oligodendrocytes, spindle neurons,
anterior lens epithelial cells, crystallin-containing lens fiber cells,
hepatocytes, adipocytes, white fat cells, brown fat cells, liver lipocytes,
kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells,
ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells,
red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands,
reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, 13 cells, natural killer cells,
melanocytes, retinal pigmented epithelial cells,
oogonia/oocytes, spermatids, spermatocytes, spermatogonium cells, spermatozoa, ovarian follicle cells, Sertoli cells, thymus epithelial cell, and/or interstitial kidney cells.

18. The FPU of claim 1, wherein cells of said at least one type of cell have been genetically engineered to produce a protein or polypeptide not naturally produced by the cell, or have been genetically engineered to produce a protein or polypeptide in an amount greater than that naturally produced by the cell, wherein said cellular composition comprises differentiated cells.

19. The FPU of claim 18, wherein said protein or polypeptide is adrenomedullin (AM), angiopoietin (Ang), bone morphogenetic protein (BMP), brain-derived neurotrophic factor (BDNF), epidermal growth factor (EGF), erythropoietin (Epo), fibroblast growth factor (FGF), glial cell line-derived neurotrophic factor (GNDF), granulocyte colony stimulating factor (G-CSF), granulocyte-macrophage colony stimulating factor (GM-CSF), growth differentiation factor (GDF-9), hepatocyte growth factor (HGF), hepatoma derived growth factor (HDGF), insulin-like growth factor (IGF), migration-stimulating factor, myostatin (GDF-8), myelomonocytic growth factor (MGF), nerve growth factor (NGF), placental growth factor (P1GF), platelet-derived growth factor (PDGF), thrombopoietin (Tpo), transforming growth factor alpha (TGF-α), TGF-β, tumor necrosis factor alpha (TNF-α), vascular endothelial growth factor (VEGF), or a Wnt protein.

20. The FPU of claim 18, wherein said protein or polypeptide is a soluble receptor for AM, Ang, BMP, BDNF, EGF, Epo, FGF, GNDF, G-CSF, GM-CSF, GDF-9 , HGF, HDGF, IGF, migration-stimulating factor, GDF-8 , MGF, NGF, PIGF, PDGF, Tpo, TGF-α, TGF-β, TNF-α, VEGF, or a Wnt protein.

21. The FPU of claim 18, wherein said protein or polypeptide is interleukin-1 alpha (IL-1α), IL-1β, IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, both IL-12 alpha and beta subunits, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23 p40 subunit, IL-23 p19 subunit and IL-23 p40 subunit together, IL-24, IL-25, IL-26, IL-27B, IL-27-p28, IL-27B and IL-27-p28 together, IL-28A, IL-28B, IL-29, IL-30, IL-31, IL-32, IL-33, IL-34, IL-35, IL-36α, IL-36β, IL-36γ.

22. The FPU of claim 18, wherein said protein or polypeptide is a soluble receptor for IL-1α, IL-1β, IL-1F1, IL-1F2, IL-1F3, IL-1F4, IL-1F5, IL-1F6, IL-1F7, IL-1F8, IL- 1F9, IL-2, IL-3, IL-4, IL-5, IL-6, IL-7, IL-8, IL-9, IL-10, IL-11, IL-12 35 kDa alpha subunit, IL-12 40 kDa beta subunit, IL-13, IL-14, IL-15, IL-16, IL-17A, IL-17B, IL-17C, IL-17D, IL-17E, IL-17F isoform 1, IL-17F isoform 2, IL-18, IL-19, IL-20, IL-21, IL-22, IL-23 p19 subunit, IL-23p40 subunit, IL-24, IL-25, IL-26, IL-27B, IL-27-p28, IL-28A, IL-28B, IL-29, IL-30, IL-31, IL-32, IL-33, IL-34, IL-35, IL-36α, IL-36β, IL-3γ.

23. The FPU of claim 18, wherein said protein or polypeptide is IFN-α, IFN-β, IFN-γ, IFN-λ1, IFN-λ2, IFN-λ3, IFN-K, IFN-ε, IFN-κ, IFN-τ, IFN-δ, IFN-λ, IFN-ω, or IFN-v.

24. The FPU of claim 18, wherein said protein or polypeptide is a soluble receptor for IFN-α, IFN-β, IFN-γ, IFN-λ1, IFN-λ2, IFN-λ3, IFN-K, IFN-ε, IFN-κ, IFN-τ, IFN-δ, IFN-λ, IFN-ω, or IFN-v.

25. The FPU of claim 18, wherein said protein or polypeptide is insulin, proinsulin, or a receptor for insulin.
